(19) United States
(12) Patent Application Publication
(10) Pub. No.: US 2003/0024003 A1
(43) Pub. Date: Jan. 30, 2003

(54) Moss genes from Physcomitrella patens encoding proteins involved in the regulation of cell division, growth and biomass formation in plants

(76) Inventors: Markus Frank, Ludwigshafen (DE); Andreas Reindl, Birkenheide (DE); Ralf-Michael Schmidt, Kirrweiler (DE); Annette Freund, Limburgerhof (DE); Thomas Ehrhardt, Speyer (DE); Friedrich Bischoff, Mannheim (DE); Andreas Renz, Limburgerhof (DE); Elke Duwenig, Freiburg (DE); Petra Cirpus, Mannheim (DE); Jens Lerchl, Ladenburg (DE); Ralf Reski, Oberried (DE)

Correspondence Address: KEIL & WEINKAUF, 1350 CONNECTICUT AVENUE, N.W., WASHINGTON, DC, 20036, US

(21) Appl. No.: 09/768,235

(22) Filed: Jan. 25, 2001

(30) Foreign Application Priority Data
Jan. 28, 2000 (EP) PCT/EP00/00675

(51) Int. Cl. A01H 5/00; C07H 21/04; C12N 9/50; C07K 14/415

(52) U.S. Cl. 800/278; 435/219; 435/410; 530/350; 536/23.4

(57) ABSTRACT

Isolated nucleic acid molecules, designated GDRP nucleic acid molecules, which encode novel GDRPs from Physcomitrella patens are described. The invention also provides antisense nucleic acid molecules, recombinant expression vectors containing GDRP nucleic acid molecules, and host cells and organisms into which the expression vectors have been introduced. The invention still further provides isolated GDRPs, mutated GDRPs, fusion proteins, antigenic peptides and methods for the improvement of production of a desired compound from transformed cells based on genetic engineering of GDRP genes in this organism.

### BACKGROUND OF THE INVENTION

From the point of yield physiology of crop plants several processes are of major concern: cell division activity, cell expansion and the onset of senescence. All these processes are tightly controlled by the action of growth factors, the so-called plant hormones. These hormones exert their function by using specific signal transduction chains and second messengers.

The phytochrome system is known to interact with the processes that regulate phytohormone balances in plant tissues. Modification of steady-state phytochrome levels in transgenic plants has been proven to display dramatic effects on growth and development.

Target genes of hormone signal transduction are most prominently cell cycle regulatory elements which play a crucial role in the control of cell divsion, growth and differentiation.

It has been shown for several examples that manipulation of the endogenous hormone level, hormone signal transduction and elements of the cell cycle regulatory machinery strongly increase plant growth, i.e. biomass formation.

### SUMMARY OF THE INVENTION

This invention provides novel nucleic acid molecules which may be used to increase biomass formation of crop plants and to modify their vegetative and reproductive development.

Given the availability of cloning vectors and techniques for genetic manipulation of several higher plant species the nucleic acid molecules of the invention may be utilized in the genetic engineering of economically important crop plants to increase yield and/or to modify agronomically interesting aspects of growth and development.

The nucleic acids of the invention encode proteins, referred to herein as Growth Development Related Proteins (GDRP). The GDRRPs are involved in the metabolism of the phytohormones auxins, brassinosteroids, cytokinins und gibberellins; the inositolphosphat-dependent signaling pathway; the regulation of the plant cell cycle; and/or the phytochrome photoreceptor system.

Thereby, the term growth development includes the improvement and/or modifications in the amount of total plant biomass leading to so-called high yield plant phaenotypes. Growth development further includes modified relations in biomass production, e.g. improved relations of apical growth and root formation.

The moss Physcomitrella patens represents one member of the mosses. It is related to other mosses such as Ceratodon purpureus which is capable to grow in the absense of light. Further Physcomitrella patens represents the only plant organism which can be utilized for targeted disruption of genes by homologous recombination. Mutants generated by this technique are useful to characterize the function for genes described in the invention. Mosses like Ceratodon and Physcomitrella share a high degree of homology on the DNA sequence and polypeptide level allowing the use of heterologous screening of DNA molecules with probes evolving from other mosses or organisms, thus enabling the derivation of a consensus sequence suitable for heterologous screening or functional annotation and prediction of gene functions in third species. The ability to identify such functions can therefore have significant relevance, e.g., prediction of substrate specificity of enzymes. Further, these nucleic acid molecules may serve as reference points for the mapping of moss genomes, or of genomes of related organisms.

Given the availability of cloning vectors for use in plants and plant transformation, such as those published in and cited therein: Plant Molecular Biology and Biotechnology (CRC Press, Boca Raton, Fla.), chapter 6/7, S.71-119 (1993); F. F. White, Vectors for Gene Transfer in Higher Plants; in: Transgenic Plants, Vol. 1, Engineering and Utilization, eds.: Kung and R. Wu, Academic Press, 1993, 15-38; B. Jenes et al., Techniques for Gene Transfer, in: Transgenic Plants, Vol. 1, Engineering and Utilization, eds.: Kung und R. Wu, Academic Press (1993), 128-143; Potrykus, Annu. Rev. Plant Physiol. Plant Molec. Biol. 42 (1991): 205-225)) the nucleic acid molecules of the invention may be utilized in the genetic engineering of a wide variety of plants to enhance biomass accumulation and/or alter important traits. This increased yield and/or altered vegetative and reproductive development may be due to a direct effect of manipulation of a gene of the invention, or it may be due to an indirect effect of such manipulation.

For example, controlled overexpression of a GDR gene encoding an enzyme which catalyzes the rate-limiting step in hormone synthesis could fundamentally enhance the level of the hormone in specific tissues at specific developmental stages. Additionally, the antisense expression of genes for enzymes involved in conjugative inactivation of phytohormones is a useful tool to increase the endogenous concentration of this hormone. Furthermore, GDR genes could by co-expressed with genes involved in the synthesis of interesting output traits in order to further increase the value of the transgenic plant.

There are a number of mechanisms by which the alteration of one of the proteins of the invention may directly affect the yield and/or plant development. Using recombinant genetic techniques well known in the art, the GDR enzymes of the invention may be manipulated such that their function is modulated. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall level of the phytohormone may be increased.

Likewise, an alteration in the regulatory features of a signal transducing protein or cell cycle regulatory protein may have a profound influence on the endogenous hormone signaling and/or mitotic activity of plant tissues. Those proteins involved in light sensing may be increased in number or activity such that photosynthesis-triggered biomass accumulation is increased.

The mutagenesis of one or more proteins of the invention may also result in proteins having altered activities which indirectly impact the production of biomass.

As increased yield is a general trait wished to be inherited into a wide variety of plants like maize, wheat, rye, oat, triticale, rice, barley, sorghum, potato, tomato, soybean, bean, pea, peanut, cotton, rapeseed, canola, alfalfa, grape, fruit plants (apple, pear, pinapple), bushy plants (coffee, cacao, tea), trees (oil palm, coconut), legumes, perennial grasses, and forage crops these crops plants are also preferred target plants for a genetic engineering as one futher embodiment of the present invention.

Accordingly, one aspect of the invention pertains to isolated GDR nucleic acid molecules (e.g., cDNAs) comprising a nucleotide sequence encoding an protein or biologically active portions thereof, as well as nucleic acid fragments suitable as primers or hybridization probes for the detection or amplification of protein encoding nucleic acid (e.g., DNA or mRNA). In particularly preferred embodiments, the isolated GDR nucleic acid molecule comprises one of the nucleotide sequences set forth in Appendix A or the coding region or a complement thereof of one of these nucleotide sequences. In other particularly preferred embodiments, the isolated nucleic acid molecule of the invention comprises a nucleotide sequence which hybridizes to or is at least about 40%, preferably at least about 50%, more preferably at least about 60%, 70% or 80%, and even most preferably at least about 90%, 95%, 99% or more homologous to a GDR nucleotide sequence set forth in Appendix A, or a portion thereof. In other preferred embodiments, the isolated nucleic acid molecule encodes one of the amino acid sequences set forth in Appendix B. The preferred GDRPs of the present invention also preferably possess at least one of the GDRP activities described herein.

In another embodiment, the isolated nucleic acid molecule encodes a protein or portion thereof wherein the protein or portion thereof includes an amino acid sequence which is sufficiently homologous to an amino acid sequence of Appendix B, e.g., sufficiently homologous to an amino acid sequence of Appendix B such that the protein or portion thereof maintains an protein activity.

Preferably, the protein or portion thereof encoded by the nucleic acid molecule maintains the ability to participate in the metabolism of compounds necessary for increasing the biomass production of plants. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (e.g., an entire amino acid sequence selected from those sequences set forth in Appendix B). In another preferred embodiment, the protein is a full length Physcomitrella patens protein which is substantially homologous to an entire amino acid sequence of Appendix B (encoded by an open reading frame shown in Appendix A).

In another preferred embodiment, the isolated nucleic acid molecule is derived from Physcomitrella patens and encodes a protein (e.g., an GDRP fusion protein) which includes a biologically active domain which is at least about 50% or more homologous to one of the amino acid sequences of Appendix B and is able to participate in the metabolism of compounds necessary for modulating the biomass production, or has one or more of the activities set forth in Table 1, and which also includes heterologous nucleic acid sequences encoding a heterologous polypeptide or regulatory regions.

Another aspect of the invention pertains to a GDRP whose amino acid sequence can be modulated with the help of art-known computer simulation programms resulting in an polypeptide with e.g. improved activity or altered regulation (molecular modelling). On the basis of this artificially generated polypeptide sequences, a corresponding nucleic acid molecule coding for such a modulated polypeptide can be synthesized in-vitro using the specific codon-usage of the desired host cell, e.g. of microorganisms, mosses, algae, ciliates, fungi or plants. In a preferred embodiment, even these artificial nucleic acid molecules coding for improved GDRPs are within the scope of this invention.

Another aspect of the invention pertains to vectors, e.g., recombinant expression vectors, containing the nucleic acid molecules of the invention, and host cells into which such vectors have been introduced, especially microorganims, plant cells, plant tissue, organs or whole plants, especially of the genus mosses or algae or crop plants.

Yet another aspect of the invention pertains to a genetically altered Physcomitrella patens plant in which an GDR protein gene has been introduced or altered. In one embodiment, the genome of the Physcomitrella patens plant has been altered by introduction of a nucleic acid molecule of the invention encoding wild-type or mutated GDR protein sequence as a transgene. In another embodiment, an endogenous protein gene within the genome of the Physcomitrella patens plant has been altered, e.g. functionally disrupted, by homologous recombination with an altered gene. In a preferred embodiment, the plant organism belongs to the genus Physcomitrella or Ceratodon, with Physcomitrella being particularly preferred.

The invention also provides an isolated preparation of an GDR protein or a portion, e.g., a biologically active portion, thereof. In a preferred embodiment, the isolated GDRP or portion thereof can participate in the metabolism of compounds necessary for the modulation of biomass production in a host cell, e.g. a microorganism or a plant cell. In another preferred embodiment, the isolated GDRP or portion thereof is sufficiently homologous to an amino acid sequence of Appendix B such that the protein or portion thereof maintains the ability to participate in the metabolism of compounds necessary for the modulation of biomass construction in microorganisms or plant cells.

The GDR polypeptide, or a biologically active portion thereof, can be operatively linked to a non-GDR polypeptide to form a fusion protein. In preferred embodiments, this fusion protein has an activity which differs from that of the GDR protein alone. In other preferred embodiment, this fusion protein performs an enzymatic reactions in hormone metabolism, signal transduction or cell cycle regulation.

The invention also provides an isolated preparation of an GDRP. In preferred embodiments, the GDRP comprises an amino acid sequence of Appendix B. In another preferred embodiment, the invention pertains to an isolated full length protein which is substantially homologous to an entire amino acid sequence of Appendix B (encoded by an open reading frame set forth in Appendix A). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B. In other embodiments, the isolated GDRP comprises an amino acid sequence which is at least about 50% or more homologous to one of the amino acid sequences of Appendix B and is able to participate in the metabolism of compounds necessary for the modulation of biomass production in a microorganism or a plant cell, or has one or more of the activities set forth in Table 1. Further, the instant invention pertains to isolated GDRPs or biologically acitve portions thereof from microorganisms, fungi, mosses, algae, plant cells, plant tissues, plant organs or whole plants.

Alternatively, the isolated GDRP can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B. It is also preferred that the preferred forms of GDRPs also have one or more of the GDRP activities described herein.

Further the instant invention pertains to an antibody specifically binding to an GDRP polypeptide mentioned before or to a portion thereof.

Another aspect of the invention pertains to a test kit comprising a nucleic acid molecule encoding a GDRP protein, a portion and/or a complement of this nucleid acid molecule used as probe or primer for identifying and/or cloning further nucleic acid molecules involved in the regulation of cell division, growth and biomass formation in other cell types or organisms.

In another embodiment the test kit comprises a GDRP-antibody for identifying and/or purifying further GDRP molecules or fragments thereof in other cell types or organisms.

Another aspect of the invention pertains to a method for producing high yield plants. This method involves either the culturing of cells, organs or whole plants containing a vector directing the expression of an GDRP nucleic acid molecule of the invention, such that a high yield plant phaenotype is produced. The method further comprises the step ot recovering the expression products selected from the group of phytohormones or photoreceptors from said cells.

In a preferred embodiment, this method further includes the step of obtaining a cell containing such a vector, in which a cell is transformed with a vector directing the expression of an GDRP nucleic acid. In a particularly preferred embodiment, the cell is a microorganism, a fungi, or belongs to the genus mosses or algae or is a plant.

Another aspect of the invention pertains to a method for producing a high yield plant which comprises the culturing of a suitable host cell whose genomic DNA has been altered by the inclusion of an GDRP nucleic acid molecule of the invention.

Another aspect of the invention pertains to methods for modulating the production of biomass from a cell. Such methods include contacting the cell with an agent which modulates GDRP activity or GDRP nucleic acid expression such that a cell associated activity is altered relative to this same activity in the absence of the agent. In a preferred embodiment, the cell is modulated for one or more metabolic pathways for phytohormones, signal transducing photoreceptors or cell cycle regulation, such that the yields or rate of production of biomass by this cell is improved. The agent which modulates GDRP activity can be an agent which stimulates GDRP activity or GDR nucleic acid expression. Examples of agents which stimulate GDRP activity or GDR nucleic acid expression include small molecules, active GDRPs, and nucleic acids encoding GDRPs that have been introduced into the cell. Examples of agents which inhibit GDRP activity or expression include small molecules and antisense GDRP nucleic acid molecules.

Another aspect of the invention pertains to methods for modulating yields of a desired compound from a cell, involving the introduction of a wild-type or mutant GDR gene into a cell, either maintained on a separate plasmid or integrated into the genome of the host cell. If integrated into the genome, such integration can be random, or it can take place by recombination such that the native gene is replaced by the introduced copy, causing the production of the desired compound from the cell to be modulated or by using a gene in trans such as the gene is functionally linked to a functional expression unit containing at least a sequence facilitating the expression of a gene and a sequence facilitating the polyadenylation of a functionally transcribed gene.

Another aspect of the invention pertains to a high yield plant produced by a method described before and the use of a high yield plant or a GDR polypeptide of the invention for the production of another high yield plant.

### DETAILED DESCRIPTION OF THE INVENTION

The present invention provides nucleic acid and protein molecules which are involved in the metabolism of phytohormones, signal transduction and cell cycle control in the moss Physcomitrella patens, referred to herein as GD (for “Growth Development”). The molecules of the invention may be utilized directly (e.g. where overexpression or optimization has a direct impact on biomass accumulation and/or development), or may have an indirect impact which nonetheless results in an increase of yield or desired changes in agronomically important traits (e.g., where modulation of the metabolism of hormones results in alterations in the root/shoot ratio). Furthermore, GDR genes could by co-expressed with genes involved in the synthesis of interesting output traits in order to further increase the value of the transgenic plant.

Preferred plants for the increase in yield and/or modulation of specific developmental aspects are the major crop plants, for example maize, wheat, rye, oat, triticale, rice, barley, sorghum, potato, tomato, soybean, bean, pea, peanut, cotton, rapeseed, canola, alfalfa, grape, fruit plants (apple, pear, pinapple), bushy plants (coffee, cacao, tea), trees (oil palm, coconut), legumes, perennial grasses, and forage crops.

The GDR gene products are implicated in the following pathways:

a.) Plant Hormone Metabolism:

i.) Cytokinins:

Cytokinins, N6-substituted adenine derivatives, are a class of plant hormones that were first identified as factors that promote cell division, and have since been implicated in many other aspects of plant growth and development including shoot initiation and growth, apical dominance, senescence and photomorphogenic development (Binns A (1994) Annu Rev Plant Physiol Plant Mol Biol 45: 173-196). Application of exogenous cytokinin to some organs that normally lack this hormone has been shown to induce cell division. Cytokinins have been linked to virtually all stages of plant development, but there has been little definitive evidence that any particular event in the cell cycle plays a role in cytokinin's induction of cell proliferation. There is an increasing body of evidence that an elevated endogenous cytokinin content increases the biomass formation (see for example Rupp et al. (1999) Plant J 18: 557-563) and/or delays the senescence (Gan S, Amasino R M (1995) Science 270: 1986-1988; WO 9629858) of transgenic plants.

The possibility that the free cytokinins are derived from tRNA has been explored extensively. Because the tRNA-bound cytokinins can act as hormonal signals for plant cells if the tRNA is degraded and fed back to the cells (Bartz J et al. (1970) Proc Natl Acad Sci USA 67(3): 1448-1455) it is likely that a significant amount of the free hormonal cytokinin in plants is derived from the turnover of tRNA. tRNA isopentenylpyrophosphate transferases are probably involved in this turnover. Therefore, consitutive, organ- or stage-specific overexpression of the tRNA isopentenylpyrophosphate transferase may provide a tool for increasing biomass and delaying senescence in transgenic plants.

ii.) Gibberellins

Gibberellins are associated with the promotion of stem length, and the application of GA to intact plants dramatically increases plant height. Moreover, they promote a wide range of developmental processes, such as photomorphogenesis, transition to flowering, promotion of seed germination and fruit set, and parthenocarpy (Hooley R (1994) Plant Mol Biol 26: 1529-1555). The major commercial uses of gibberellins are in the management of fruit crops, the malting of barley, and the extension of sugarcane, with a resulting increase in sugar yield. In some crops, a reduction in height is desirable, and this can be accomplished by the use of gibberellin synthesis inhibitors (Gianfanga T J (1995) In: Plant Hormones: Physiology, Biochemistry and Molecular Biology, P J Davies, ed., pp. 751-773).

An organ- or stage-specific transgenic manipulation of the endogenous GA content has not been carried out so far. Given the availability of different promoter systems, the nucleic acids of the invention encoding key enzymes of gibberellin biosynthesis, copalylpyrophosphate synthase, ent-kaurene synthase and gibberelin 3 beta-hydroxylase, provide an efficient tool for alteration of the endogenous gibberellin concentration and accumulation of particular metabolites (EP 692537, WO 9535383).

iii.) Auxins

Auxins promote plant cell division, elongation and differentiation. Beside these effects auxins promote the apical dominance and lateral root formation (Evans M L (1985) Crit Rev Plant Sci 2: 213-265). Conjugative and degradative mechanisms are used to regulate IAA levels in plants. Amide-linked conjugates of indole-3-acetic acid (IAA) are putative storage or inactivation forms of auxin. Genes encoding enzymes which are involved in the conversion of free bases into conjugates and vice versa are essential tools for the manipulation of the endogenous concentration of biologically active auxin metabolites.

One gene of the invention encodes an UDP-glucose: indole 3-acetate-beta-D-glucosyltransferase. This enzyme catalyzes the first step in the biosynthesis of IAA conjugates (Szerszen J B et al. (1994) Science 265:1699-701). The second GDR gene of the invention related to auxin metabolism encodes an IM amino acid hydrolase which participates in the release of IAA from amino acid conjugates (Bartel B and Fink G R (1995) Science 268: 1745-1748).

iii.) Brassinosteroids

Many steroids have been identified in plants, but only the brassinosteroids had been shown to cause marked biological effects on plant growth at very low concentrations, and to be widely distributed throughout the plant kingdom (Mandava N B (1988) Annu Rev plant Physiol Plant Mol Biol 39: 23-52). Physiological studies have established that exogenous brassinosteroid causes cell elongation and cell division in excised stem sections. Brassionsteroids also inhibit root growth and delay leaf abscission. Brassinosteroids are derived from the plant sterol campersterol by a reduction step followed by many oxidation steps (Fujioka M and Sakurai A (1997) Physiol Plantarum 100: 710-715). Two GDR genes encode enzymes which participate in the metabolism of brassinosteroids, i.e. a C-4 methyl sterol oxidase (Bard M et al. (1996) Proc Natl Acad Sci USA 93(1): 186-190) and a C-24 methyltransferase (Grebenok R J et al. (1997) Plant Mol Biol 34(6): 891-896). These genes provide useful tools to transgenically elevate the endogenous brassinosteroid levels thereby increasing growth and biomass formation of crop plants.

b.) Cell Cycle Regulators:

There is extensive literature regarding the regulation of the cell cycle in yeast, animal cells and to a certain extent in plants (Jacobs T W (1995) Annu Rev Plant Physiol Plant Mol Biol 46: 329-339). Cell cycle progression is controlled at the G1/S and G2/M checkpoints, primarily by two classes of proteins: cyclins, and cyclin dependent kinases (CDKs). Passage through the checkpoints require the activation of CDKs, and this is achieved by association with cyclins and by altering the phosphorylation state of the CDK. By associating with different cyclins, cdc2, the first CDK identified, controls both G1/S and G2/M transition in yeast. B-type cyclins are the major class of mitotic phase-specific cyclins which act at the G2/M transition, as it has also been shown for the A-type cyclins. The D-type cyclins are the major class involved in G1/S transition. cDNAs encoding CDKs and G1 and mitotic cyclins have been isolated in plants, and CDK inhibitors have been shown to block cell cycle progression at both G1/S and G2/M transition in Arabidopsis and Petunia cells (Glab N et al. (1994) FEBS Left 353: 207-211).

It has previously been shown that the constitutitve overexpression of cyclin B1 in Arabidopsis roots leads to an increased biomass (Doerner P et al. (1996) Nature 380: 520-523). Similarly, overexpression of an altered cdc2a in tobacco plants yielded plants which exhibited significantly more biomass than untransformed control plants (Hemerly A et al. (1995) EMBO J 14: 3925-3936). Most strikingly, transgenic expression of the Cyclin D2 gene under the transcriptional control of the CaMV 35S promoter resulted in plants with a shortened plastochron, bigger leaves and inflorescences. In contrast, cks1, which in vitro binds to Cdc2, acts as an inhibitor of cell divisions and appears to promote endoreduplication (De Veylder L et al. (1997) FEBS Lett 412 (3): 446-452). Taken together, these results underline the potential of the manipulation of the plant cell cycle to increase biomass of crop plants.

One gene of the invention encodes an A-type cylin (Setiady Y Y et al. (1995) Plant J 8 (6): 949-957), a second gene has significant amino acid identity to G1/S-specific cyclins (Cross FR (1990) Moll Cell Biol 10 (12): 6482-6490). Another gene of the invention encodes a cyclin G-associated kinase that is also involved in the control of cell cycle progression (Kanaoka Y et al. (1997) FEBS Left 402 (1): 73-80).

c.) Inositolphosphate-Dependent Pathway

Calcium serves as a second messenger for a wide variety of cell signaling events, such as phytochrome and gibberellin signaling. In plant cells, most of the calcium accumulates in the vacuole. Certain hormones can induce the release from intracellular compartments by the opening of intracellular calcium channels. The coupling of hormone binding to the opening of intracellular calcium channels is mediated by the second messenger inositol trisphosphate (Bethke PC et al. (1995) In: Plant Hormones: Physiology, Biochemistry and Molecular Biology, pp. 298-317). Phosphatidylinositol (PI) is a minor phospholipid component of cell membranes. PI can be converted to the polyphosphoinositides PI phosphate (PIP) and PI bisphosphate (PIP₂) by kinases. In animal cells, binding of a hormone, such as vasopressin, to its receptor leads to the activation of heterotrimeric G proteins. The a subunit then dissociates from G and activates a phosphoinositide-specific phospholipase, phospholipase C (PLC). The activated PLC rapidly hydrolyzes PIP₂, generating inositol trisphosphate (IP₃) and diacylglycerol (DAG) as products. Each of these molecules plays an important role in cell signaling. Two nucleic acids of the invention encode inositol monophosphatases which are involved in the conversion of IP₃ to inositol and other intermediates of the inositolphosphate-dependent signaling pathway (Gillaspy G E et al. (1995) Plant Cell 7 (12): 2175-2185) and provide, therefore, tools to manipulate Ca²⁺-dependent developmental processes. In plants, this signal transduction pathway could be correlated with the action of plant growth regulators, e.g. gibberellins and auxins (Scherer G F (1995) Biochem Soc Trans 23(4): 871-875). It is also implicated in phytochrome signaling (Neuhaus G et al. (1993) Cell 73: 937-952). Accordingly, manipulation of the IP₃-signal transduction pathway provide a useful tool for the modification of plant growth and development.

d.) Phytochrome:

Phytochrome is the best characterized photoreceptor in plants. It exists in plants in two spectrally distinct forms; the Pr form that absorbs maximally in the red (λmax=666 nm) region of the spectrum and the Pfr form that absorbs maximally in the far-red (λmax=730 nm) region of the spectrum. Pr is reversibly converted to Pfr by absorbing far-red light. In vivo, photoconversion of Pr to Pfr by red light induces a vast array of morphogenic responses whereas reconversion of Pfr back to Pr by far-red light represses the induction of the responses.

Phytochrome has been proven to be functionally involved in the control of developmental processes as deetiolation of germinating seedlings, hypocotyl elongation and cotyledon expansion, regulation of the synthesis of plastid proteins (e.g. of the photosynthetic apparatus), control of shade tolerance, and transition to flowering and fruit production.

Altering phytochrome levels in light-grown plants will provide a opportunity to influence many developmental processes throughout the life cycle of the plant. Modification of the steady-state phytochrome concentrations in the cells of light-grown plants may result in the creation of a number of desirable growth and developmental traits that would have agronomic benefit. Furthermore, the phytochrome system is known to regulate phytohormone balances in plant tissues. Alterations of steady-state phytochrome levels in transgenic plants may change the balances between the various endogenous phytohormones (i.e. gibberellins, brassinosteroids, cytokinins and auxins) that govern plant growth and development, and thereby give rise to plants with new traits which have agronomic value (EP 0354 687).

Elements and Methods of the Invention

The present invention is based, at least in part, on the discovery of novel molecules, referred to herein as GDR (for “Growth Development Related”) nucleic acid and protein molecules, which play a role in or function in one or more cellular metabolic and regulatory pathways in Physcomitrella patens. In a particularly preferred embodiment, the GDR proteins encoded by GDR nucleotides of the invention are modulated in activity, such that the metabolic and regulatory pathways which the GDR proteins of the invention involve are modulated.

The language, GDR protein or GDR polypeptide includes proteins which play a role in, e.g., catalyze an enzymatic reaction, in metabolic and/or regulatory pathways that are implicated in the control of plant growth and development. Examples of GDR proteins include those encoded by the GDR genes set forth in Table 1 and Appendix A. The terms GDR gene or GDR nucleic acid sequence include nucleic acid sequences encoding an GDR protein, which consist of a coding region and also corresponding untranslated 5′ and 3′ sequence regions. Examples of GDR genes include those set forth in Table 1. The term yield is art-recognized and includes the total amount of biomass. The terms biosynthesis or a biosynthetic pathway are art-recognized and include the synthesis of a compound, preferably an organic compound, by a cell from intermediate compounds in what may be a multistep and highly regulated process. The terms degradation or a degradation pathway are art-recognized and include the breakdown of a compound, preferably an organic compound, by a cell to degradation products (generally speaking, smaller or less complex molecules) in what may be a multistep and highly regulated process. The language metabolism is art-recognized and includes the totality of the biochemical reactions that take place in an organism. The metabolism of a particular compound, then, (e.g., the metabolism of a hormone) comprises the overall biosynthetic, modification, and degradation pathways in the cell related to this compound.

The mutagenesis of one or more GDR genes of the invention may also result in GDR proteins having altered activities which indirectly influence plant growth and development. For example, a biosynthetic enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired compound without impairing the viability of the cell. In each case, the overall plant growth may be increased or altered.

The isolated nucleic acid sequences of the invention are contained within the genome of a Physcomitrella patens strain available through the moss collection of the University of Hamburg. The nucleotide sequence of the isolated Physcomitrella patens GDR cDNAs and the predicted amino acid sequences of the respective Physcomitrella patens GDR proteins are shown in Appendices A and B, respectively.

The present invention also pertains to proteins which have an amino acid sequence which is substantially homologous to an amino acid sequence of Appendix B. As used herein, a protein which has an amino acid sequence which is substantially homologous to a selected amino acid sequence is least about 30% identical to the selected amino acid sequence, e.g., the entire selected amino acid sequence

Various aspects of the invention are described in further detail in the following subsections:

A. Isolated Nucleic Acid Molecules

One aspect of the invention pertains to isolated nucleic acid molecules that encode GDR polypeptides or biologically active portions thereof, as well as nucleic acid fragments sufficient for use as hybridization probes or primers for the identification or amplification of GDR protein-encoding nucleic acid (e.g., GDR DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated GDR nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, a Physcomitrella patens cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, or a portion thereof, can be isolated using standard molecular biology techniques and the sequence information provided herein. For example, a P. patens GDR cDNA can be isolated from a P. patens library using all or portion of one of the sequences of Appendix A as a hybridization probe and standard hybridization techniques (e.g., as described in Sambrook et al., Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, mRNA can be isolated from plant cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an GDR nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A. The sequences of Appendix A correspond to the Physcomitrella patensGDR cDNAs of the invention. This cDNA comprises sequences encoding GDR proteins (i.e., the “coding region”, indicated in each sequence in Appendix A), as well as 5′ untranslated sequences and 3′ untranslated sequences. Alternatively, the nucleic acid molecule can comprise only the coding region of any of the sequences in Appendix A or can contain whole genomic fragments isolated from genomic DNA. For the purposes of this application, it will be understood that each of the sequences set forth in Appendix A has an identifying entry number Each of these sequences comprises up to three parts: a 5′ upstream region, a coding region, and a downstream region. Each of these three regions is identified by the same entry number designation to eliminate confusion. The recitation one of the sequences in Appendix A, then, refers to any of the sequences in Appendix A, which may be distinguished by their differing entry number designations. The coding region of each of these sequences is translated into a corresponding amino acid sequence, which is set forth in Appendix B. The sequences of Appendix B are identified by the same entry numbers designations as Appendix A, such that they can be readily correlated. For example, the amino acid sequence in Appendix B designated 54_ppprot1_{—}52 is a translation of the coding region of the nucleotide sequence of nucleic acid molecule 54_ppprot1_{—}52 in Appendix A, and the amino acid sequence in Appendix B designated 38_ck7_g07fwd is a translation of the coding region of the nucleotide sequence of nucleic acid molecule 38_ck7_g07fwd in Appendix A.

In another preferred embodiment, an isolated nucleic acid molecule of the invention comprises a nucleic acid molecule which is a complement of one of the nucleotide sequences shown in Appendix A, or a portion thereof. A nucleic acid molecule which is complementary to one of the nucleotide sequences shown in Appendix A is one which is sufficiently complementary to one of the nucleotide sequences shown in Appendix A such that it can hybridize to one of the nucleotide sequences shown in Appendix A, thereby forming a stable duplex.

In an additional preferred embodiment, an isolated nucleic acid molecule of the invention comprises a nucleotide sequence which hybridizes, e.g., hybridizes under stringent conditions, to one of the nucleotide sequences shown in Appendix A, or a portion thereof.

Moreover, the nucleic acid molecule of the invention can comprise only a portion of the coding region of one of the sequences in Appendix A, for example a fragment which can be used as a probe or primer or a fragment encoding a biologically active portion of an GDR protein. The nucleotide sequences determined from the cloning of the GDR genes from P. patens allows for the generation of probes and primers designed for use in identifying and/or cloning GDR protein homologues in other cell types and organisms, as well as GDR protein homologues from other mosses or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone GDR protein homologues. Probes based on the GDR nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which misexpress an GDR protein, such as by measuring a level of an GDR protein-encoding nucleic acid in a sample of cells, e.g., detecting GDR mRNA levels or determining whether a genomic GDR gene has been mutated or deleted.

Portions of proteins encoded by the GDR nucleic acid molecules of the invention are preferably biologically active portions of one of the GDR protein. As used herein, the term “biologically active portion of an GDR protein” is intended to include a portion, e.g., a domain/motif, of an GDR protein that participates in the metabolism of hormones, in signal transduction or cel cycle regulation.

Additional nucleic acid fragments encoding biologically active portions of an GDR protein can be prepared by isolating a portion of one of the sequences in Appendix B, expressing the encoded portion of the GDR protein or peptide (e.g., by recombinant expression in vitro) and assessing the activity of the encoded portion of the GDR protein or peptide.

The invention further encompasses nucleic acid molecules that differ from one of the nucleotide sequences shown in Appendix A (and portions thereof) due to degeneracy of the genetic code and thus encode the same GDR protein as that encoded by the nucleotide sequences shown in Appendix A. In another embodiment, an isolated nucleic acid molecule of the invention has a nucleotide sequence encoding a protein having an amino acid sequence shown in Appendix B

In addition to the Physcomitrella patens GDR nucleotide sequences shown in Appendix A, it will be appreciated by those skilled in the art that DNA sequence polymorphisms that lead to changes in the amino acid sequences of GDR proteins may exist within a population (e.g., the Physcomitrella patens population). Such genetic polymorphism in the GDR gene may exist among individuals within a population due to natural variation. As used herein, the terms “gene” and “recombinant gene” refer to nucleic acid molecules comprising an open reading frame encoding an GDR protein, preferably a Physcomitrella patens GDR protein. Such natural variations can typically result in 1-5% variance in the nucleotide sequence of the GDR gene. Any and all such nucleotide variations and resulting amino acid polymorphisms in GDR proteins that are the result of natural variation and that do not alter the functional activity of GDR proteins are intended to be within the scope of the invention. Nucleic acid molecules corresponding to natural variants and non-Physcomitrella patens homologues of the Physcomitrella patens GDR cDNA of the invention can be isolated based on their homology to Physcomitrella patens GDR nucleic acid disclosed herein using the Physcomitrella patens cDNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to a sequence of Appendix A corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural Physcomitrella patens GDR protein.

In addition to naturally-occurring variants of the GDR protein sequence that may exist in the population, the skilled artisan will further appreciate that changes can be introduced by mutation into a nucleotide sequence of Appendix A, thereby leading to changes in the amino acid sequence of the encoded GDR protein, without altering the functional ability of the GDR protein. For example, nucleotide substitutions leading to amino acid substitutions at “non-essential” amino acid residues can be made in a sequence of Appendix A. A “non-essential” amino acid residue is a residue that can be altered from the wild-type sequence of one of the GDR proteins (Appendix B) without altering the activity of said GDR protein, whereas an “essential” amino acid residue is required for GDR protein activity. Other amino acid residues, however, (e.g., those that are not conserved or only semi-conserved in the domain having GDR protein activity) may not be essential for activity and thus are likely to be amenable to alteration without altering GDR protein activity.

Accordingly, another aspect of the invention pertains to nucleic acid molecules encoding GDR proteins that contain changes in amino acid residues that are not essential for GDR protein activity. Such GDR proteins differ in amino acid sequence from a sequence contained in Appendix B yet retain at least one of the GDR protein activities described herein.

To determine the percent homology of two amino acid sequences (e.g., one of the sequences of Appendix B and a mutant form thereof or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B) is occupied by the same amino acid residue or nucleotide as the corresponding position in the other sequence (e.g., a mutant form of the sequence selected from Appendix B), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=numbers of identical positions/total numbers of positions×100).

An isolated nucleic acid molecule encoding an GDR protein homologous to a protein sequence of Appendix B can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an GDR protein is preferably replaced with another amino acid residue from the same side chain family. Alternatively, in another embodiment, mutations can be introduced randomly along all or part of an GDR protein coding sequence, such as by saturation mutagenesis, and the resultant mutants can be screened for an GDR protein activity described herein to identify mutants that retain GDR protein activity. Following mutagenesis of one of the sequences of Appendix A, the encoded protein can be expressed recombinantly and the activity of the protein can be determined using, for example, assays described herein (see Example 17 of the Exemplification).

In addition to the nucleic acid molecules encoding GDR proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire GDR cDNA coding strand, or to only a portion thereof. In one embodiment, an antisense nucleic acid molecule is antisense to a “coding region” of the coding strand of a nucleotide sequence encoding an GDR protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding GDR proteins. The term “noncoding region” refers to 5′ and 3′ sequences which flank the coding region that are not translated into amino acids (i.e., also referred to as 5′ and 3′ untranslated regions).

Given the coding strand sequences encoding GDR proteins disclosed herein (e.g., the sequences set forth in Appendix A), antisense nucleic acids of the invention can be designed according to the rules of Watson and Crick base pairing. The antisense nucleic acid molecule can be complementary to the entire coding region of GDR mRNA, but more preferably is an oligonucleotide which is antisense to only a portion of the coding or noncoding region of GDR mRNA. For example, the antisense oligonucleotide can be complementary to the region surrounding the translation start site of GDR mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an GDR protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic including plant promoters are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Left. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave GDR mRNA transcripts to thereby inhibit translation of GDR mRNA. A ribozyme having specificity for an GDR protein-encoding nucleic acid can be designed based upon the nucleotide sequence of an MP protein cDNA disclosed herein. For example, a derivative of a Tetrahymena L-1-9 IVS RNA can be constructed in which the nucleotide sequence of the active site is complementary to the nucleotide sequence to be cleaved in an GDR protein-encoding mRNA. See, e.g., Cech et al. U.S. Pat. No. 4,987,071 and Cech et al. U.S. Pat. No. 5,116,742. Alternatively, GDR mRNA can be used to select a catalytic RNA having a specific ribonuclease activity from a pool of RNA molecules. See, e.g., Bartel, D. and Szostak, J. W. (1993) Science 261:1411-1418.

Alternatively, GDR gene expression can be inhibited by targeting nucleotide sequences complementary to the regulatory region of an GDR nucleotide sequence (e.g., an GDR promoter and/or enhancers) to form triple helical structures that prevent transcription of an GDR gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

B. Recombinant Expression Vectors and Host Cells

Another aspect of the invention pertains to vectors, preferably expression vectors, containing a nucleic acid encoding an MP protein (or a portion thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence are fused to each other so that both sequences fulfil the proposed function addicted to the sequence used. (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990) or in.Gruber and Crosby, in: Methods in Plant Molecular Biology and Biotechnolgy, CRC Press, Boca Raton, Fla., eds.:Glick and Thompson, Chapter 7, 89-108 including the references therein. Regulatory sequences include those which direct constitutive expression of a nucleotide sequence in many types of host cell and those which direct expression of the nucleotide sequence only in certain host cells or under certain conditions. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GDR proteins, mutant forms of GDR proteins, fusion proteins, etc.).

The recombinant expression vectors of the invention can be designed for expression of GDR proteins in prokaryotic or eukaryotic cells. For example, GDR genes can be expressed in bacterial cells such as E. coli, insect cells (using baculovirus expression vectors), yeast and other fungal cells (see Romanos, M. A. et al. (1992) Foreign gene expression in yeast: a review, Yeast 8: 423-488; van den Hondel, C. A. M. J. J. et al. (1991) Heterologous gene expression in filamentous fungi, in: More Gene Manipulations in Fungi, J. W. Bennet & L. L. Lasure, eds., p. 396-428: Academic Press: San Diego; and van den Hondel, C. A. M. J. J. & Punt, P. J. (1991) Gene transfer systems and vector development for filamentous fungi, in: Applied Molecular Genetics of Fungi, Peberdy, J. F. et al., eds., p. 1-28, Cambridge University Press: Cambridge), algae (Falciatore et al., 1999, Marine Biotechnology.1 (3):239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in WO9801572 and multicellular plant cells (see Schmidt, R. and Willmitzer, L. (1988), High efficiency Agrobacterium tumefaciens-mediated transformation of Arabidopsis thaliana leaf and cotyledon explants, Plant Cell Rep.: 583-586); Plant Molecular Biology and Biotechnology, C Press, Boca Raton, Florida, chapter 6/7, S.71-119 (1993); F. F. White, B. Jenes et al., Techniques for Gene Transfer, in: Transgenic Plants, Vol. 1, Engineering and Utilization, eds.:Kung und R. Wu, Academic Press (1993), 128-43; Potrykus, Annu. Rev. Plant Physiol. Plant Molec. Biol. 42 (1991), 205-225; or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the GDR protein is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant GDR protein unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128).

Another strategy is to alter the nucleic acid sequence of the nucleic acid to be inserted into an expression vector so that the individual codons for each amino acid are those preferentially utilized in the bacterium chosen for expression, such as E. coli. Such alteration of nucleic acid sequences of the invention can be carried out by standard DNA synthesis techniques.

In another embodiment, the GDR protein expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge.

Alternatively, the GDR proteins of the invention can be expressed in insect cells using baculovirus expression vectors. Baculovirus vectors available for expression of proteins in cultured insect cells (e.g., Sf 9 cells) include the pAc series (Smith et al. (1983) Mol. Cell Bio. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the fetoprotein promoter (Campes and Tilghman (1989) Genes Dev. 3:537-546).

In another embodiment, the GDR proteins of the invention may be expressed in unicellular plant cells (such as algae) see Falciatore et al., 1999, Marine Biotechnology.1 (3):239-251 and references therein and plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation, Nucl. Acid. Res. 12: 8711-8721; Vectors for Gene Transfer in Higher Plants; in: Transgenic Plants, Vol. 1, Engineering and Utilization, eds.: Kung und R. Wu, Academic Press, 1993, S. 15-38.

A plant expression cassette preferably contains regulatory sequences capable to drive gene expression in plants cells and which are operably linked so that each sequence can fulfil its function such as termination of transcription such as polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3 (1984), 835 ff) or functional equivalents therof but also all other terminators are suitable.

As plant gene expression is very often not limited on transcriptional levels a plant expression cassette preferably contains other operably linked sequences like translational enhancers such as the overdrive-sequence containing the 5′-untranlated leader sequence from tobacco mosaic virus enhancing the protein per RNA ratio (Gallie et al 1987, Nucl. Acids Research 15:8693-8711).

Plant gene expression has to be operably linked to an appropriate promoter conferring gene expression in a timely, cell or tissue specific manner. Preferrred are promoters driving constitutitive expression (Benfey et al., EMBO J. 8 (1989) 2195-2202) like those derived from plant viruses like the 35S CAMV (Franck et al., Cell 21(1980) 285-294), the 19S CaMV (see also U.S. Pat. No. 5,352,605 and WO8402913) or plant promoters like those from Rubisco small subunit described in U.S. Pat. No. 4,962,028. WO 8705629, WO 9204449.

Other preferred sequences for use operable linkage in plant gene expression cassettes are targeting-sequences necessary to direct the gene-product in its appropriate cell compartment (for review see Kermode, Crit. Rev. Plant Sci. 15, 4 (1996), 285-423 and references cited therin) such as the vacuole, the nucleus, all types of plastids like amyloplasts, chloroplasts, chromoplasts, the extracellular space, mitochondria, the endoplasmic reticulum, oil bodies, peroxisomes and other compartments of plant cells.

Plant gene expression can also be facilitated via a chemically inducible promoter (for rewiew see Gatz 1997, Annu. Rev. Plant Physiol. Plant Mol. Biol., 48:89-108). Chemically inducible promoters are especially suitable if gene expression is wanted to occur in a time specific manner. Examples for such promoters are a salicylic acid inducible promoter (WO 95/19443), a tetracycline inducible promoter (Gatz et al., (1992) Plant J. 2, 397-404) and an ethanol inducible promoter (WO 93/21334).

Also promoters responding to biotic or abiotic stress conditions are suitable promoters such as the pathogen inducible PRP1-gene promoter (Ward et al., Plant. Mol. Biol. 22 (1993), 361-366), the heat inducible hsp8o-promoter from tomato (U.S. Pat. No. 5,187,267), cold inducible alpha-amylase promoter from potato (WO9612814) or the wound-inducible pinll-promoter (EP375091).

Especially those promoters are preferred which confer gene expression in storage tissues and organs such as cells of the endosperm and the developing embryo. Suitable promoters are the napin-gene promoter from rapeseed (U.S. Pat. No. 5,608,152), the USP-promoter from Vicia faba (Baeumlein et al., Mol Gen Genet, 1991, 225 (3):459-67), the oleosin-promoter from Arabidopsis (WO9845461), the phaseolin-promoter from Phaseolus vulgaris (U.S. Pat. No. 5,504,200), the Bce4-promoter from Brassica (WO9113980) or the legumin B4 promoter (LeB4; Baeumlein et al., 1992, Plant Journal, 2 (2):233-9) as well as promoters conferring seed specific expression in monocot plants like maize, barley, wheat, rye, rice etc. Suitable promoters to note are the Ipt2 or Ipt1-gene promoter from barley (WO9515389 and WO9523230) or those desribed in WO9916890 (promoters from the barley hordein-gene, the rice glutelin gene, the rice oryzin gene, the rice prolamin gene, the wheat gliadin gene, wheat glutelin gene, the maize zein gene, the oat glutelin gene, the Sorghum kasirin-gene, the rye secalin gene).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to GDR mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986 and Mol et al., 1990, FEBS Letters 268:427-430.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an GDR protein can be expressed in bacterial cells such as E. coli, insect cells, fungal cells or mammalian cells (such as Chinese hamster ovary cells (CHO) or COS cells), algae, ciliates, plant cells or fungi. Other suitable host cells are known to those skilled in the art.

Vector DNA can be introduced into prokaryotic or eukaryotic cells via conventional transformation or transfection techniques. As used herein, the terms “transformation” and “transfection”, conjugation and transduction are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells including plant cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989) and other laboratory manuals such as Methods in Molecular Biology, 1995, Vol. 44, Agrobacterium protocols, ed: Gartland and Davey, Humana Press, Totowa, N.J.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate or in plants that confer resistance towards a herbicide such as glyphosate or glufosinate. Nucleic acid encoding a selectable marker can be introduced into a host cell on the same vector as that encoding an GDR protein or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an GDR gene into which a deletion, addition or substitution has been introduced to thereby alter, e.g., functionally disrupt, the GDR gene. Preferably, this GDR gene is a Physcomitrella patens GDR gene, but it can be a homologue from a related plant or even from a mammalian, yeast, or insect source. In a preferred embodiment, the vector is designed such that, upon homologous recombination, the endogenous GDR gene is functionally disrupted (i.e., no longer encodes a functional protein; also referred to as a knock-out vector). Alternatively, the vector can be designed such that, upon homologous recombination, the endogenous GDR gene is mutated or otherwise altered but still encodes functional protein (e.g., the upstream regulatory region can be altered to thereby alter the expression of the endogenous GDR protein). To create a point mutation via homologous recombination also DNA-RNA hybrids can be used known as chimeraplasty known from Cole-Strauss et al. 1999, Nucleic Acids Research 27(5):1323-1330 and Kmiec Gene therapy. 19999, American Scientist. 87(3):240-247.

Whereas in the homologous recombination vector, the altered portion of the GDR gene is flanked at its 5′ and 3′ ends by additional nucleic acid of the GDR gene to allow for homologous recombination to occur between the exogenous GDR gene carried by the vector and an endogenous GDR gene in a microorganism or plant. The additional flanking GDR nucleic acid is of sufficient length for successful homologous recombination with the endogenous gene. Typically, several hundreds of basepairs up to kilobases of flanking DNA (both at the 5′ and 3′ ends) are included in the 3s vector (see e.g., Thomas, K. R., and Capecchi, M. R. (1987) Cell 51: 503 for a description of homologous recombination vectors or Strepp et al., 1998, PNAS, 95 (8):4368-4373 for cDNA based recombination in Physcomitrella patens). The vector is introduced into a microorganism or plant cell (e.g., via polyethyleneglycol mediated DNA) and cells in which the introduced GDR gene has homologously recombined with the endogenous GDR gene are selected, using art-known techniques.

In another embodiment, recombinant microorganisms can be produced which contain selected systems which allow for regulated expression of the introduced gene. For example, inclusion of an GDR gene on a vector placing it under control of the lac operon permits expression of the GDR gene only in the presence of IPTG. Such regulatory systems are well known in the art.

A host cell of the invention, such as a prokaryotic or eukaryotic host cell in culture, can be used to produce (i.e., express) an GDR protein. An alternate method can be applied in addition in plants by the direct transfer of DNA into developing flowers via electroporation or Agrobacterium medium gene transfer. Accordingly, the invention further provides methods for producing GDR proteins using the host cells of the invention. In one embodiment, the method comprises culturing the host cell of invention (into which a recombinant expression vector encoding an GDR protein has been introduced, or into which genome has been introduced a gene encoding a wild-type or altered GDR protein) in a suitable medium until GDR protein is produced. In another embodiment, the method further comprises isolating GDR proteins from the medium or the host cell.

C. Isolated GDR proteins

Another aspect of the invention pertains to isolated GDR proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of GDR protein in which the protein is separated from cellular components of the cells in which it is naturally or recombinantly produced. In one embodiment, the language “substantially free of cellular material” includes preparations of GDR protein having less than about 30% (by dry weight) of non-GDR protein (also referred to herein as a “contaminating protein”), more preferably less than about 20% of non-GDR protein, still more preferably less than about 10% of non-GDR protein, and most preferably less than about 5% non-GDR protein. When the GDR protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of GDR protein in which the protein is separated from chemical precursors or other chemicals which are involved in the synthesis of the protein. In one embodiment, the language “substantially free of chemical precursors or other chemicals” includes preparations of GDR protein having less than about 30% (by dry weight) of chemical precursors or non-GDR protein chemicals, more preferably less than about 20% chemical precursors or non-GDR protein chemicals, still more preferably less than about 10% chemical precursors or non-GDR protein chemicals, and most preferably less than about 5% chemical precursors or non-GDR protein chemicals. In preferred embodiments, isolated proteins or biologically active portions thereof lack contaminating proteins from the same organism from which the GDR protein is derived. Typically, such proteins are produced by recombinant expression of, for example, a Physcomitrella patens GDR protein in other plants than Physcomitrella patens or microorganisms such as E. coli or ciliates, algae or fungi.

GDR proteins are preferably produced by recombinant DNA techniques. For example, a nucleic acid molecule encoding the protein is cloned into an expression vector (as described above), the expression vector is introduced into a host cell (as described above) and the GDR protein is expressed in the host cell. The GDR protein can then be isolated from the cells by an appropriate purification scheme using standard protein purification techniques. Alternative to recombinant expression, an MP protein, polypeptide, or peptide can be synthesized chemically using standard peptide synthesis techniques. Moreover, native GDR protein can be isolated from cells (e.g., endothelial cells), for example using an anti-GDR protein antibody, which can be produced by standard techniques utilizing an GDR protein or fragment thereof of this invention.

The invention also provides GDR protein chimeric or fusion proteins. As used herein, an GDR “chimeric protein” or “fusion protein” comprises an GDR polypeptide operatively linked to a non-GDR polypeptide. An “GDR polypeptide” refers to a polypeptide having an amino acid sequence corresponding to an GDR protein, whereas a “non-GDR polypeptide” refers to a polypeptide having an amino acid sequence corresponding to a protein which is not substantially homologous to the GDR protein, e.g., a protein which is different from the GDR protein and which is derived from the same or a different organism. Within the fusion protein, the term “operatively linked” is intended to indicate that the GDR polypeptide and the non-GDR polypeptide are fused to each other so that both sequences fulfil the proposed function addicted to the sequence used. The non-GDR polypeptide can be fused to the N-terminus or C-terminus of the GDR polypeptide. For example, in one embodiment the fusion protein is a GST-GDR fusion protein in which the GDR protein sequences are fused to the C-terminus of the GST sequences. Such fusion proteins can facilitate the purification of recombinant GDR proteins. In another embodiment, the fusion protein is an GDR protein containing a heterologous signal sequence at its N-terminus. In certain host cells (e.g., mammalian host cells), expression and/or secretion of an GDR protein can be increased through use of a heterologous signal sequence.

Preferably, an GDR chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An GDR protein-encoding nucleic acid can be cloned into such an expression vector such that the fusion moiety is linked in-frame to the GDR protein.

Homologues of the GDR protein can be generated by mutagenesis, e.g., discrete point mutation or truncation of the MP protein. As used herein, the term “homologue” refers to a variant form of the GDR protein which acts as an agonist or antagonist of the activity of the GDR protein. An agonist of the GDR protein can retain substantially the same, or a subset, of the biological activities of the GDR protein. An antagonist of the GDR protein can inhibit one or more of the activities of the naturally occurring form of the GDR protein, by, for example, competitively binding to a downstream or upstream member of the cell membrane component metabolic cascade which includes the GDR protein, or by binding to an GDR protein which mediates transport of compounds across such membranes, thereby preventing translocation from taking place.

In an alternative embodiment, homologues of the GDR protein can be identified by screening combinatorial libraries of mutants, e.g., truncation mutants, of the GDR protein for GDR protein agonist or antagonist activity. In one embodiment, a variegated library of GDR protein variants is generated by combinatorial mutagenesis at the nucleic acid level and is encoded by a variegated gene library. A variegated library of GDR protein variants can be produced by, for example, enzymatically ligating a mixture of synthetic oligonucleotides into gene sequences such that a degenerate set of potential GDR protein sequences is expressible as individual polypeptides, or alternatively, as a set of larger fusion proteins (e.g., for phage display) containing the set of GDR protein sequences therein. There are a variety of methods which can be used to produce libraries of potential GDR protein homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential GDR protein sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the GDR protein coding can be used to generate a variegated population of GDR protein fragments for screening and subsequent selection of homologues of an GDR protein. In one embodiment, a library of coding sequence fragments can be generated by treating a double stranded PCR fragment of an GDR protein coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the GDR protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of GDR protein homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify GDR protein homologues (Arkin and Yourvan (1992) PNAS 89:7811-7815; Delgrave et al. (1993) Protein Engineering 6(3):327-331).

In another embodiment, cell based assays can be exploited to analyze a variegated GDR protein library, using methods well known in the art.

D. Uses and Methods of the Invention

The nucleic acid molecules, proteins, protein homologues, fusion proteins, primers, vectors, and host cells described herein can be used in one or more of the following methods: identification of Physcomitrella patens and related organisms; mapping of genomes of organisms related to Physcomitrella patens; identification and localization of Physcomitrella is patens sequences of interest; evolutionary studies; determination of GDR protein regions required for function; modulation of an GDR protein activity.

The GDR nucleic acid molecules of the invention have a variety of uses. First, they may be used to identify an organism as being Physcomitrella patens or a close relative thereof. Also, they may be used to identify the presence of Physcomitrella patens or a relative thereof in a mixed population of microorganisms. The invention provides the nucleic acid sequences of a number of Physcomitrella patens genes; by probing the extracted genomic DNA of a culture of a unique or mixed population of microorganisms under stringent conditions with a probe spanning a region of a Physcomitrella patens gene which is unique to this organism, one can ascertain whether this organism is present.

Further, the nucleic acid and protein molecules of the invention may serve as markers for specific regions of the genome. This has utility not only in the mapping of the genome, but also for functional studies of Physcomitrella patens proteins. For example, to identify the region of the genome to which a particular Physcomitrella patens DNA-binding protein binds, the Physcomitrella patens genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of Physcomitrella patens, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related mosses, such as Physcomitrella patens.

The GDR nucleic acid molecules of the invention are also useful for evolutionary and protein structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the GDR nucleic acid molecules of the invention may result in the production of GDR proteins having functional differences from the wild-type GDR proteins. These proteins may be improved in efficiency or activity, may be present in greater numbers in the cell than is usual, or may be decreased in efficiency or activity.

This invention is further illustrated by the following examples which should not be construed as limiting. The contents of all references, patent applications, patents, and published patent applications cited throughout this application are hereby incorporated by reference.

### EXAMPLIFICATION

### Example 1

### General Processes

a) General Cloning Processes:

Cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA were carried out as described in Sambrook et al. (1989) (Cold Spring Harbor Laboratory Press: ISBN 0-87969-309-6) or Kaiser, Michaelis and Mitchell (1994), Methods in Yeasr Genetics” (Cold Spring Harbor Laboratory Press: ISBN 0-87969-451-3). Transformation and cultivation 21 of algae such as Chlorella or Phaeodactylum are transformed as described by El-Sheekh (1999), Biologia Plantarum 42: 209-216; Apt et al. (1996), Molecular and General Genetics 252 (5): 872-9.

b) Chemicals:

The chemicals used were obtained, if not mentioned otherwise in the text, in p.a. quality from the companies Fluka (Neu-Ulm), Merck (Darmstadt), Roth (Karlsruhe), Serva (Heidelberg) and Sigma (Deisenhofen). Solutions were prepared using purified, pyrogen-free water, designated as H₂O in the following text, from a Milli-Q water system water purification plant (Millipore, Eschborn). Restriction endonucleases, DNA-modifying enzymes and molecular biology kits were obtained from the companies AGS (Heidelberg), Amersham (Braunschweig), Biometra (Göttingen), Boehringer (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Amsterdam, Netherlands). They were used, if not mentioned otherwise, according to the manufacturer's instructions.

c) Plant Material:

For this study, plants of the species Physcomitrella patens (Hedw.) B.S.G. from the collection of the genetic studies section of the University of Hamburg were used. They originate from the strain 16/14 collected by H.L.K. Whitehouse in Gransden Wood, Huntingdonshire (England), which was subcultured from a spore by Engel (1968, Am J Bot 55, 438-446). Proliferation of the plants was carried out by means of spores and by means of regeneration of the gametophytes. The protonema developed from the haploid spore as a chloroplast-rich chloronema and chloroplast-low caulonema, on which buds formed after approximately 12 days. These grew to give gametophores bearing antheridia and archegonia. After fertilization, the diploid sporophyte with a short seta and the spore capsule resulted, in which the meiospores mature.

d) Plant Growth:

Culturing was carried out in a climatic chamber at an air temperature of 25□C and light intensity of 55 micromols-1m-2 (white light; Philips TL 65W/25 fluorescent tube) and a light/dark change of 16/8 hours. The moss was either modified in liquid culture using Knop medium according to Reski and Abel (1985, Planta 165, 354-358) or cultured on Knop solid medium using 1% oxoid agar (Unipath, Basingstoke, England). The protonemas used for RNA and DNA isolation were cultured in aerated liquid cultures. The protonemas were comminuted every 9 days and transferred to fresh culture medium.

### Example 2

### Total DNA Isolation from Plants

The details for the isolation of total DNA relate to the working up of one gram fresh weight of plant material.

CTAB buffer: 2% (w/v) N-cethyl-N,N,N-trimethylammonium bromide (CTAB); 100 mM Tris HCl pH 8.0; 1.4 M NaCl; 20 mM EDTA.

N-Laurylsarcosine buffer: 10% (w/v) N-laurylsarcosine; 100 mM Tris HCl pH 8.0; 20 mM EDTA.

The plant material was triturated under liquid nitrogen in a mortar to give a fine powder and transferred to 2 ml Eppendorf vessels. The frozen plant material was then covered with a layer of 1 ml of decomposition buffer (1 ml CTAB buffer, 100 ml of N-laurylsarcosine buffer, 20 ml of b-mercaptoethanol and 10 ml of proteinase K solution, 10 mg/ml) and incubated at 60□C for one hour with continuous shaking. The homogenate obtained was distributed into two Eppendorf vessels (2 ml) and extracted twice by shaking with the same volume of chloroform/isoamyl alcohol (24:1). For phase separation, centrifugation was carried out at 8000× g and RT for 15 min in each case. The DNA was then precipitated at −70□C for 30 min using ice-cold isopropanol. The precipitated DNA was sedimented at 4□C and 10,000 g for 30 min and resuspended in 180 ml of TE buffer (Sambrook et al., 1989, Cold Spring Harbor Laboratory Press: ISBN 0-87969-309-6). For further purification, the DNA was treated with NaCl (1.2 M final concentration) and precipitated again at −70□C for 30 min using twice the volume of absolute ethanol. After a washing step with 70% ethanol, the DNA was dried and subsequently taken up in 50 ml of H₂O+RNAse (50 mg/ml final concentration). The DNA was dissolved overnight at 4□C and the RNAse digestion was subsequently carried out at 37□C. for 1 h. Storage of the DNA took place at 4□C.

### Example 3

### Isolation of Total RNA and Poly-(A)+ RNA from Plants

For the investigation of transcripts, both total RNA and poly-(A)⁺ RNA were isolated. The total RNA was obtained from wild-type 9d old protonemata following the GTC-method (Reski et al. 1994, Mol. Gen. Genet., 244:352-359).

Isolation of PolyA+ RNA was isolated using Dyna Beads^{R} (Dynal, Oslo) Following the instructions of the manufacturers protocol.

After determination of the concentration of the RNA or of the poly-(A)+ RNA, the RNA was precipitated by addition of 1/10 volumes of 3 M sodium acetate pH 4.6 and 2 volumes of ehanol and stored at −70□C.

### Example 4

### cDNA Library Construction

For cDNA library construction first strand synthesis was achieved using Murine Leukemia Virus reverse transcriptase (Roche, Mannheim, Germany) and olido-d(T)-primers, second strand synthesis by incubation with DNA polymerase I , Klenow enzyme and RNAseH digestion at 12° C. (2h), 16° C. (1h) and 22° C. (1h). The reaction was stopped by incubation at 65° C. (10 min) and subsequently transferred to ice. Double stranded DNA molecules were blunted by T4-DNA-polymerase (Roche, Mannheim) at 37° C. (30 min). Nucleotides were removed by phenol/chloroform extraction and Sephadex-G50 spin columns. EcoRI adapters (Pharmacia, Freiburg, Germany) were ligated to the cDNA ends by T4-DNA-ligase (Roche, 12° C., overnight) and phosphorylated by incubation with polynucleotide kinase (Roche, 37° C., 30 min). This mixture was subjected to separation on a low melting agarose gel. DNA molecules larger than 300 basepairs were eluted from the gel, phenol extracted, concentrated on Elutip-D-columns (Schleicher and Schuell, Dassel, Germany) and were ligated to vector arms and packed into lambda ZAPII—phages or lambda ZAP-Express phages using the Gigapack Gold Kit (Stratagene, Amsterdam, Netherlands) using material and following the instructions of the manufacturer.

### Example 5

### Identification of Genes of Interest

Gene sequences can be used to identify homologous or heterologous genes from cDNA or genomic libraries.

Homologous genes (e.g. full length cDNA clones) can be isolated via nucleic acid hybridization using for example cDNA libraries: Depended on the abundance of the gene of interest 100 000 up to 1 000 000 recombinant bacteriophages are plated and transferred to a nylon membrane. After denaturation with alkali, DNA is immobilized on the membrane by e.g. UV cross linking. Hybridization is carried out at high stringency conditions. In aqueous solution hybridization and washing is performed at an ionic strength of 1 M NaCl and a temperature of 68□C. Hybridization probes are generated by e.g. radioactive (³²p) nick transcription labeling (Amersham Ready Prime). Signals are detected by exposure to x-ray films.

Partially homologous or heterologous genes that are related but not identical can be identified analog to the above described procedure using low stringency hybridization and washing conditions. For aqueous hybridization the ionic strength is normally kept at 1 M NaCl while the temperature is progressively lowered from 68 to 42□C.

Isolation of gene sequences with homologies only in a distinct domain of (for example 20 aminoacids) can be carried out by using synthetic radio labeled oligonucleotide probes. Radio labeled oligonucleotides are prepared by phosphorylalation of the 5′-prime end of two complementary oligonucleotides with T4 polynucleotede kinase. The complementary oligonucleotides are annealed and ligated to form concatemers. The double stranded concatemers are than radiolabled by for example nick transcription. Hybridization is normally performed at low stringency conditions using high oligonucleotide concentrations.

Oligonucleotide hybridization solution:

6× SSC

0.01 M sodium phosphate

1 mM EDTA (pH 8)

0.5% SDS

100 μg/ml denaturated salmon sperm DNA

0.1% nonfat dried milk

During hybridization temperature is lowered stepwise to 5-10 □C below the estimated oligonucleotid Tm.

Further details are described by Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons.

### Example 6

### Identification of Genes of Interest by Screening Expression Libraries with Antibodies

C-DNA sequences can be used to produce recombinant protein for example in E. coli (e.g. Qiagen QIAexpress pQE system). Recombinant proteins are than normally affinity purified via Ni-NTA affinity chromatoraphy (Qiagen). Recombinant proteins are than used to produce specific antibodies for example by using standard techniques for rabbit immunization. Antibodies are affinitypurified using a Ni-NTA column saturated with the recombinant antigen as described by Gu et al., (1994) BioTechniques 17: 257-262. The antibody can than be used to screen expression cDNA libraries to identify homologous or heterologous genes via an immunological screening (Sambrook, J. et al. (1989), “Molecular Cloning: A Laboratory Manual”, Cold Spring Harbor Laboratory Press or Ausubel, F. M. et al. (1994) “Current Protocols in Molecular 3s Biology”, John Wiley & Sons).

### Example 7

### Northern-Hybridization

For RNA hybridization, 20 mg of total RNA or 1 mg of poly-(A)+ RNA were separated by gel electrophoresis in 1.25% strength agarose gels using formaldehyde as described in Amasino (1986, Anal. Biochem. 152, 304), transferred by capillary attraction using 10× SSC to positively charged nylon membranes (Hybond N+, Amersham, Braunschweig), immobilized by UV light and prehybridized for 3 hours at 68° C. using hybridization buffer (10% dextran sulfate w/v, 1 M NaCl, 1% SDS, 100 mg of herring sperm DNA). The labeling of the DNA probe with the “Highprime DNA labeling kit” (Roche, Mannheim, Germany) was carried out during the prehybridization using alpha-³²P dCTP (Amersham, Braunschweig, germany). Hybridization was carried out after addition of the labeled DNA probe in the same buffer at 68° C. overnight. The washing steps were carried out twice for 15 min using 2× SSC and twice for 30 min using 1× SSC, 1% SDS at 68° C. The exposure of the sealed-in filters was carried out at −70° C. for a period of 1-14d.

### Example 8

### DNA Sequencing

C-DNA libraries as described in Example 4 were used for DNA sequencing according to standard methods, in particular by the chain termination method using the ABI PRISM Big Dye Terminator Cycle Sequencing Ready Reaction Kit (Perkin-Elmer, Weiterstadt, germany). Random Sequencing was carried out subsequent to preparative plasmid recovery from cDNA libraries via in vivo mass excision and retransformation of DH10B on agar plates (material and protocol details from Stratagene, Amsterdam, Netherlands. Plasmid DNA was prepared from overnight grown E. coli cultures grown in Luria-Broth medium containing ampicillin (see Sambrook et al. (1989) (Cold Spring Harbor Laboratory Press: ISBN 0-87969-309-6)) on a Qiagene DNA preparation robot (Qiagen, Hilden) according to the manufacturers protocols. Sequencing primers with the following nucleotide sequences were used:

5′-CAGGAAACAGCTATGACC-3′
5′-CTAAAGGGAACAAAAGCTG-3′
5′-TGTAAAACGACGGCCAGT-3′

### Example 9

### Plasmids for Plant Transformation

For plant transformation binary vectors such as pBinAR can be used (Höfgen and Willmitzer, Plant Science 66(1990), 221-230). Construction of the binary vectors can be performed by ligation of the cDNA in sense or antisense orientation into the T-DNA.

5′-prime to the cDNA a plant promotor activates transcription of the cDNA. A polyadenylation sequence is located 3′-prime to the cDNA. Tissue specific expression can be archived by using a tissue specific promotor. For example seed specific expression can be archived by cloning the napin or USP promotor 5-prime to the cDNA. Also any other seed specific promotor element can be used. For constitutive expression within the whole plant the CaMV 35S promotor can be used.

The expressed protein can be targeted to a cellular compartment using a signal peptide, for expample for plasids, mitochondria or endoplasmatic reticulum (Kermode, Crit. Rev. Plant Sci. 15, 4 (1996), 285-423). The signal peptide is cloned 5′-prime in frame to the cDNA to archive subcellular localization of the fusionprotein.

Nucleic acid molecules from Physcomitrella are used for a direct gene knock-out by homologous recombination. Therefore Physcometrella sequences are useful for functional genomic approaches. The technique is described by Strepp et al., Proc. Natl. Acad. Sci. USA, 1998, 95: 4369-4373; Girke et al. (1998), Plant Journal 15: 39-48; Hofmann et al. (1999) Molecular and General Genetics 261: 92-99.

### Example 10

### Transformation of Agrobacterium

Agrobacterium mediated plant transformation can be performed using for example the GV3101(pMP90) (Koncz and Schell, Mol. Gen.Genet. 204 (1986), 383-396) or LBA4404 (Clontech) Agrobacterium tumefaciens strain. Transformation can be performed by standard transformation techniques (Deblaere et al., Nucl. Acids. Tes. 13 (1984), 4777-4788).

### Example 11

### Plant Transformation

Agrobacterium mediated plant transformation has been performed using standard transformation and regeneration techniques (Gelvin, Stanton B.; Schilperoort, Robert A, “Plant Molecular Biology Manual”,2nd Ed.—Dordrecht: Kluwer Academic Publ., 1995.—in Sect., Ringbuc Zentrale Signatur: BT11-P ISBN 0-7923-2731-4; Glick, Bernard R.; Thompson, John E., “Methods in Plant Molecular Biology and Biotechnology”, Boca Raton: CRC Press, 1993.-360 S.,ISBN 0-8493-5164-2).

For example rapeseed can be transformed via cotyledon or hypocotyl transformation (Moloney et al., Plant cell Report 8 (1989), 238-242; De Block et al., Plant Physiol. 91 (1989, 694-701). Use of antibiotica for agrobacterium and plant selection depends on the binary vector and the agrobacterium strain used for transformation. Rapeseed selection is normally performed using kanamycin as selectable plant marker.

Agrobacterium mediated gene transfer to flax can be performed using for example a technique described by Mlynarova et al. (1994), Plant Cell Report 13: 282-285.

Transformation of soybean can be performed using for example a technique described in EP 0424 047, U.S. Pat. No. 322,783 (Pioneer Hi-Bred International) or in EP 0397 687, U.S. Pat. No. 5,376,543, U.S. Pat. No. 5,169,770 (University Toledo).

Plant transformation using particle bombardment, Polyethylene Glycol mediated DNA uptake or via the Silicon Carbide Fiber technique is for example described by Freeling and Walbot “The maize handbook” (1 993) ISBN 3-540-97826-7, Springer Verlag N.Y.).

### Example 12

### In vivo Mutagenesis

In vivo mutagenesis of microorganisms can be performed by passage of plasmid (or other vector) DNA through E coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 2277-2294, ASM: Washington.) Such strains are well known to those skilled in the art. The use of such strains is illustrated, for example, in Greener, A. and Callahan, M. (1994) Strategies 7: 32-34. Transfer of mutated DNA molecules into plants is preferably done after selection and testing in microorganisms. Transgenic plants are generated according to various examples within the exemplification of this document.

### Example 13

### Assessment of the Expression of a Recombinant Gene Product in a Transformed Organism

The activity of a recombinant gene product in the transformed host organism has been measured on the transcriptional or/and on the translational level.

A useful method to ascertain the level of transcription of the gene (an indicator of the amount of mRNA available for translation to the gene product) is to perform a Northern blot (for reference see, for example, Ausubel et al. (1988) Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the transformed gene. Total cellular RNA can be prepared from cells, tissues or organs by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or colorimetric label which may be readily detected. The presence and quantity of label observed indicates the presence and quantity of the desired mutant protein present in the cell.

### Example 14

### In vitro Analysis of the Function of Physcomitrella Genes in Transgenic Organisms

The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: New York; Walsh, (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1994) Enzymkinetik, 2^{nd} ed. VCH: Weinheim (ISBN 3527300325); Bergmeyer, H. U., Bergmeyer, J., Graβl, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

Equivalents

Those skilled in the art will recognize, or will be able to ascertain using no more than routine experimentation, many equivalents to the specific embodiments of the invention described herein. Such equivalents are intended to be encompassed by the following claims.

Legends to the Figurs:

Table 1: Enzymes involved in the modulation of improved production of biomass and the accession/entry number of the corresponding nucleic acid molecule

Appendix A: Nucleic acid sequences encoding for GDRP polypeptides

Appendix B: GDRP polypeptide sequences

TABLE 1
FIGURES

| | | Start of | Stop of |
|---|---|---|---|
| | | open | open |
| | | reading | reading |
| Function | Acc. no./Entry no. | frame | frame |
| Cell cycle control | | | |
| A-type Cyclin | 54_ppprot1_52 | 1-3 | 100-102 |
| G1/S-specific Cyclin | 38_ck7_g07fwd | 1-3 | 199-201 |
| CDC39 | 48_ck9_h09fwd | 1-3 | 532-534 |
| CDC48 | 83_ck6_f06fwd | 4-6 | 541-543 |
| CDC48 | 06_mm09_a09rev | 1-3 | 460-462 |
| CDC2 Kinase | 71_ck31_d06fwd | 1-3 | 265-267 |
| Cks1At | 54_ppprot1_062_a09 | 1-3 | 229-231 |
| Cytokinin metabolism | | | |
| tRNA delta 2-Isopentenyl-pyrophosphate | 06_ppprot1_062_a09 | 1-3 | 448-450 |
| Transferase | | | |
| Steroid metabolism | | | |
| C4 Sterol Methyl Oxidase | 36_ck24_f09fwd | 1-3 | 430-432 |
| C4 Sterol Methyl Oxidase | 02_ppprot1_087_a07 | 1-3 | 529-531 |
| C24 Sterol Methyltransferase | 26_ppprot1_054_e07 | 1-3 | 604-606 |
| Inositolephosphate-dependent Pathway | | | |
| Inositole Monophosphatase | 88_ppprot135_g11 | 1-3 | 424-426 |
| Inositole Monophosphatase | 87_mm12_g05rev | 1-3 | 361-363 |
| Phytochrome | | | |
| Phytochrome | 53_mm18_a06rev | 1-3 | 505-507 |
| Gibberellin metabolism | | | |
| Copalylpyrophosphate Synthase | 93_ck24_h05fwd | 1-3 | 481-483 |
| Ent-kaurene Synthase | 51_ppprot1_0052_a05 | 1-3 | 259-261 |
| Ent-kaurene Synthase | 38_ppprot1_046_g07 | 1-3 | 622-624 |
| Ent-kaurene Synthase | 66_ppprot1_63 | 1-3 | 145-1 47 |
| Gibberellin-3 beta Hydroxylase | 48_ppprot1_063_h09 | 1-2 | 397-399 |
| Auxin metabolism | | | |
| IAA Hydrolase | 73_bd05_e04rev | 1-3 | 400-402 |
| Auxin resistance protein homologue | 47_bd01_h03rev | 1-3 | 376-376 |

Appendix A

Cell cycle control
54_ppprot1_52
CCCATACGAGAGAAATACAGAAATCATAAGTTCAAGTGTGTGGCAACGTT
GACGCCTCCTTCAGTACTTCCCCCGGAATTTTTCAAAGACGCTGAATGCT
GC
38_ck7g07fwd
ACAAATAGAGATGTTAACTACGACATTACTCCAGTCACTGCACTATGTGT
GGGAACGGCAACATGGGAATCTTGTAACAAAACTATGACAGTCAAAACTG
GAAGTGACCTGGTATGCCGACATAGATACCACTCAAAAAAATACGCTGAA
ATCAAGACAAGCGAAAGCCTCCTTCGTACAAAGGAGCGTCCGAAGTCAAC
TACACCACAT
48_ck9_h09fwd
GGCACGAGCGTGCCTCTCCTTAACGCGCTGGTGCTTTACGTTGGAATGCA
GGCTGTTCAGCAACTTCATAGCAAGACATCGCAGCAGTTGGCAGCACTCA
CGGCTCCTATTACTCACAGCGCTCCAATGGACATATTTCAGCGACTCGTG
AATGATCTCGACACAGAAGGAAGATATCTCTTTTTGAACGCTGTGGCAAA
TCAACTGCGCTACCCCAACAATCATACATATTACTTCTCGTGTGTACTTC
TGTTCCTCTTTGCGGAGGCTTCACTTGAAATCATTCAGGAACAGATTACT
CGTGTGTTGCTCGAGACACTAATTGTCAATCGGCCACACCCTTGGGGTCT
GCTTATCACCTTTATTGAGCTTATCAAGAATCCACGCTACAGCTTTTGGA
CACACGGCTTCACTCGATGTGCTCCAGAAATTGACAAGCTATTTGAGTCA
GTGGCCCGTTCATGCATGAATTCTACTTTGAAGCCCAGTGATGACGATCT
GCCCGGAAACCTTCCAGCTGACGGTTTGAAAGGA
83_ck6_f06fwd
CNTGATATTATAGACTCTGCACTACTTACGCCTGGACGTTTGGATCAGCT
CATTTATATTCCTCTACCAGATGAAGCTTCTCGCTTGAGAATTTTCCAAG
CAGCTTTAAGGAAGAGTCCTTTGGCCAAGGAGGTGGATTTGGAAGCCCTG
GCCAGGTACACACAAGGTTTCAGTGGTGCGGATATCACTGAAATCTGTCA
GCGCGCTTGCAAGTACGCCATTCGTGAAAACATTGAGAAGGATATTGAGA
GGGAAAAGAGAATGGCAGAGAATCCCGAAGCTATGGAAGAAGACGAGGTA
GAAGAAGTTGCGCAGATCAAGGCATCCCACTTTGAAGAAGCAATGAAATA
TGCTCGCCGAAGTGTAAGTGATGCTGACATCCGCAAGTACCAAGCGTTTG
CTCAGACTCTGCAGCAGTCGCGTGGATTCGGATCGGAGTTTCGATTCCCA
GATCGTGCAGTTGGTGCAGGAGCACCAAGCGCTGCCGACACTACTCCGGG
ATTTGGTGTAGCAGCTGCAGCTGATGATGATGATTTGTATAGC
06_mm09_a09rev
CACCAGGAGCATCCTGAGAAGTTTGAGAAGTTTGGAATGTCGCCGTCTAA
GGGTGTATTGTTCTATGGTCCGCCTGGGTGTGGAAAGACGCTGCTCGCCA
AGGCAATTGCCAATGAATGTCAAGCCAATTTTATCAGTGTGAAGGGGCCC
GAGCTGTTGACCATGTGGTTCGGTGAGAGTGAAGCGAATGTGCGAGATGT
ATTTGACAAGGCTCGTCAGTCAGCTCCTTGTGTTCTCTTTTTCGATGAGC
TTGACTCGATCGCCAATCAGCGTGGCAGCAGTCAGGGTGATGCTGGTGGT
GCTGCGGATCGTGTCTTAAACCAGCTGCTTACAGAGATGGATGGTATGAA
CGCCAAAAAGACGGTGTTCATCATTGGGGCAACTAATAGACCTGATATTA
TAGACTCTGCACTTCTCAGACCTGGACGTTTGGGATCAGCTCATTTACAT
TCCCCTNCCNGA
71_ck3l_d06fwd
GCACGAGCCGAACTTCAGCAGCTTCTTCACATCTTCAGGTTGCTTGGCAC
CCCGAATGAGACAATCTGGCCTGGTGTTAGCCAGCACCGTGATTGGCACG
AGTTTCCTCAATGGAGACCACAAGATCTGTCCCTTGCTGTTCCCGGACTC
AGCGCGGTTGGCTTAGACCTTCTCGCCAAAATGTTGGTATTCGAGCCCTC
AAAGAGAATCTCTGCCAAAGCCGCCTTGAGCCATACTTATTTCGCTGATG
TTGATAAGACAGCAACC
54_ppprot1._062_a09
TCTCGNTACCTCAAGTTTCAAATGGATCANTATGAGAAAGTGGAGAAGAT
TGGAGAGGGCACATNCGGTGTCGTATACAAGGCCCGGGATCGCCTCACTA
ATGAAACTATTGCTCTGAAAAAAATACGGCTGGAGCAAGAAGATGAAGGT
GTTCCAAGCNCCGCCATTCGANAAATTTCACTCCTGAAAGAAATGCACCA
TGGCAACATCGTTCGGCTACAAGATGTGGTG
Cytokinin metabolism
06_ppprot1_062_a09
TTTCTGATGGAGTGTCGTCAAGTGGAAGGCGCGGCCACAGAAGAGCGATT
TTTTCTTTTTCTAGAGGAATTCCAACGACACTCCAGGAATTATGTCAAAA
GGCAGTTAACATGGTTCCGAAATAAAGGTCAAAGTGAGCAGATGTTCAAC
TGGATTGATGCCACACAGCCCCTAGAAGTGATGGTGGACGCCTTAGCGAA
AGAGTATGAAAGGCCCAATGAAGTGGTGAGCGATGTCCTGAAAGCGGCAA
GTGTTGTTACCAAGGAGTCTAGTTACAAGGAGGAAAACCTTTTGAAGCGC
TACCGAACTCAAAACAGGATATTTACTAGTAACAGTGAGGCGCTCAAGCG
TACTTTACAATGGATACGAGATACCCAGTGTCTATGGCGGAACAGTAGCA
CGGTGGATGATCTCCAAAAGAGAATGGAATCATCCTTCACGACCTCTATG
Steroid metabolism
36_ck24_f09fwd
ATAGAGAGAGAGAGAGAGAGAGAGAGAGAGAGACAGGAATTAACTCCCGC
GTGGGGTGCGGTGTCGACCTCGTCGATCAGCATGGCGACTGCTCTGGAGA
AAGGATGGCTGTACCTAATTAACAACTTCACTGACTTCCAGCTGGCATCT
ATTGGCAGTTTCATTATTCACGAGAGTGTGTTCTTCCTTTCTGGTCTTCC
ATTCATTGTTATGCAGACATTGGGTTACCAAAGGCAGTACAAGATACAGG
GAAAGGTTAATTCTGTTGCAGCACAGGAGAAGTGTGTTATGAAGCTTTTG
ATTTATCACATCTGCGTCAATTTGCCTCTAATGATCGTGTCATATCCTGT
CTTCAAATACATGGGCTTCACCAGTCAATTACCTCTACCTTCCTGGAATG
TAGTGTGCTTTCAGATCTATCTTATTTTATCT
02_ppprot1_087_a07
GCACGAGGTTCCTTCCCTGCCGTGAGGTTGATAGGGGTTCGGTTCGGTCT
GCCCCTGCCGGCTGTCAGTGAGGTGTTGATGCAGCTTACTGTGTATACCA
TAGTTGAGGATTTCGGGAACTATTGGTTGCATCGCTGGTTGCACAATGGT
TGGTGGTACGATGCCATTCATTCTGTGCACCATGAGTTCTCCGCGCCGAT
GAGCTTCGCCGCCCCTTATGCACATTGGGCGGAGGTTGTGATTTTAGGGG
TTCCTACCTTCGCGGGTCCAGCGATGGCGCCTGGCCATATCATCACCTTC
TGGCTTTGGATTGCAATGAGGCAGTTGGAGGCTCTCGAAACTCACAGCGG
ATACGATTTCCCTTGGAACCCCACGCGCTTGATTCCCTTCTACGGAGGGG
CAGAGTATCACGACTATCACCACTTCGTCGGCGCTAAGTGTTCAAGCAAC
TTCGCCTCTGTTTTCACGTACTGCGACTGGCTATACGGCACCGATAAGGG
ATACCGNTACATGAAGGAGGTGCACAGGAAA
26_ppprot1_054_e07
GCACGAGGCGGGGTGAAGCATCTCGCCCCTGCCTGCTCTCATTCTCATGC
TCCGCCCCGCTTTTCGCTGCTAAATCCTTGCGCCTCCACTGTGTGCTCCC
CTGGTCTTGAATACGGGTTTAGTGGGTGGGTTCCTTCACACTGCTTTCCA
CGCCCCACTCTGTTTCATCGACCGAGAGTTGTGTGGCACTTCACTGCAGC
AGCTCTTTGCGTGGTCCTGTTTCCCTCGGCGTCCACAGAGAAGTTGGCAG
ATATGGCGAAGGAAGGGGCGAGGCATTTGGTGAGCAACATTGGCGGAGTT
TTGCCGAAACATGAGGTCCTCAAGTCGACTGAAGAGTACGAGAAGTATCA
TGCTATGCACGGAGGCGACAAAGAAGCAAGAAAGAGCAATTACACTGACA
TGGTGAACAAGTACTACGACCTTGTTAACAGCTTCTACGAGTATGGGTGG
GGAGAGTCTTACCACTTTGCCAACAGATGGCGCGGGGAAACGCTTCGTGA
AAGCATTAAACGACACGAGCATTTTCTGGCTTTGCATCTTCATTTGAAAC
CAGGAATGAAGGTGTTGGATGTCGGGTGTGGAATTGGTGGTCCTGCTCGT
GGAATC
Inositolephosphate-dependent Pathway
88_ppprot135_g11
GTTGCGATCCGAGAGCTGAGTCAACTGAGAACTCAAGGGTCGTGCCCTCC
GAAGGACGGCTTCACCAACATGGGCCTTCAGCAGTGTGAGGATCTGGAGA
CCTGCTTAGCAGTTGCAGTGGACGTTGCTAAGAAAGCGGGGCAGATCATC
AAGGATGGTTTCCACATAGCGAAGGCTGTCCAACACAAGGGCATGGTGGA
TCTCGTCACCGAGACGGACAAGGCCTGTGAGGACTTGATCTTCACGCAGC
TCAAGACATCATTTCCGTCCCATCAGTTAATAGGAGAGGAGGAATCTTCC
GAAAGCGGAATTCCACTCTTGACAGATGCGCCGACTTGGGTGGTTGACCC
CCTGGACGGCACCACAAATTTCGTGCACAAATTTCCCTTTGTGTGCGTCT
CCATTGGTCTCGTCATAAACAAGGTC
87_mm12_g05rev
CACGAGCAAACGAACCTTGAACTATTCAAGTACTACACAGACACTAGTCG
GGGTGTCAGGAGATTGGGTGCAGCTGCGGTAGACTGTTGTCACGTTGCTC
TTGGTATTGCTGATTCTTACTGGGAATTTCGCCTCAAACCCTGGGATATG
GCTGCTGGTGCTCTGATGGTAGAAGAGGCTGGAGGAAAGGTGACGCGTAT
GGATGGTGGGCCTTTCTCTGTCTTTGATCGTTCTGTCATCGTCAGCAACG
GAGCTATTCATGACAAGCTGTTGGAAAAGATTCAACCCGCCACCGAGAAG
CTCATTGCGGATGGATTGAACTTTTCCCAATGGTTGAAGCCCACTGGCTA
TAACTCAGATGTG
Phytochrome
53_mm18_a06rev
GCACCAGTAAATGATTTTCGGAATAAAAAATATGCTCCTGGGGCGGTCAC
TCCATTTTCAATCACACAAGCGGTTGACCTGATGCTGCAGCTTGCTGAAG
GCGTGAGATACCTTCACAGCAAGCACCTGGCTCACCGTGACATCAAGTCG
GGCAATGTTCTTCTACAATTCGCGGACCCTAAACACGGAACGACAGAACC
ATGGAGCAATGGTAACACCTGTCCCTTCATTGCAAAGGTGGCAGACTTTG
GGTTGACAAAGATTAAGAACACTAGCACGCACAGGGGTCATCAAACACTT
ATGACAGGCACTAGGCCTTGGATGGCTCCAGAGGCTTACAAGTATGAATG
GACAGATGAACCCACACCGTCCTCTCGCTACCACCCCATGAAACTCGATG
TTTATGGATTTGGAATTATGTGCTGCGAAATATTGTCAGGGGAGGAGCCA
TACCAGAAATTACCGTCGTATGCTGCTGTAAAGGCCGGAGAGAGGCCCGA
AGTTGCC
Gibberellin metabolism
93_ck24_h05fwd
GGCACGAGCGACTACTTGAACCAGCTCCTCATCAAGTTCGACCACGCTTG
TCCAAACGTGTACCCCGTTGATCTCTTCGAGCGTTTGTGGATGGTAGACC
GCCTACAAAGGCTGGGAATATCCCGCTACTTCGAGCGAGAAATCAGAGAC
TGTCTACAATATGTATACCGATACTGGAAGGATTGTGGTATTGGCTGGGC
AAGCAATTCGTCCGTGCAGGACGTGGACGACACGGCCATGGCCTTCCGCC
TTCTCCGCACACACGGATTCGACGTCAAGGAGGACTGCTTCAGACACTTT
TTCAAAGATGGTGAGTTCTTCTGCTTCGCCGGCCAGTCCAGCCAAGCCGT
CACGGGAATGTTCAACCTCAGCAGAGCATCGCAAACGCTCTTCCCAGGGG
AATCACTCCTAAAAAAGGCCANAACCTTTTCCAGAAACTTTTTGAGAACC
AAGCATGAAAACAATGAATGCTTCGACAAGTGG
51_ppprot1_0052_a05
AAGAGAGAAGAAAATGAAAAAAGCAGGATTCCTATGGCGATGGTGTACAA
GTACCCCACTACTTTGCTGCATTCTCTGGAAGGCCTGCACCGGGAAGTGG
ACTGGAACAAGCTCCTCCAGCTACAGTCCGAGAATGGCTCCTTTCTGTAT
TCACCCGCATCCACTGCATGCGCACTTGTACACAAAAGATGTGAAGTGCT
TCGACTACTTGAACCAGCTCCTCATCAAGTTCGACCACGCTTGTCCAAAC
GTGTACCCCGT
38_ppprot1_046_g07
GCACGAGGTCTTGAGCATCGCACCTACTTCGACCAATATGGGATTGATGA
TATCTGGATTGGCAAGTCGCTCTACAAAATGCCGGCCGTCACCAACGAAG
TGTTTCTCAAATTGGCCAAAGCCGACTTCAACATGTGCCAAGCTCTTCAC
AAGAAGGAACTCGAGCAGGTCATCAAATGGAATGCCAGCTGCCAATTTAG
AGACCTCGAGTTTGCTAGACAGAAATCCGTGGAGTGCTACTTCGCAGGCG
CTGCAACCATGTTTGAGCCCGAAATGGTGCAGGCGAGGCTCGTTTGGGCA
CGCTGTTGCGTGCTCACCACCGTTCTACACGATTACTTCGATCACGGTAC
ACCTGTGGAAGAGCTTCGGGTTTTTGTGCAGGCCGTAAGGACTTGGAATC
CCGAGCTCATCAACGGACTACCTGAGCAAGCCAAGATTCTCTTTATGGGA
CTGTACAAGACTGTGAACACTATCGCCGAGGAGGCATTCATGGCACAGAA
ACGAGACGTACATCATCATCTCAAGCATTACTGGGACAAATTGATCACTT
CAGCTTTGAAAG0AGCCCGAATGGGCAGAGTCCGGCTACGTTCCCCACCT
TCGACGAGTATATGGAAGTCGCTG
66_ppprot1_63
TTCACTGCAGTCCCGAAGTCCTGTAAGAGAATCCATTTAAACATGGCGAA
GATCATGCACGCTTTCTACAAGGACACTGATGGGTTTTCGTCACTGACAG
CCATGACAGGGTTTGTGAAGAAGGTGCTCTTCGAGCCAGTACCTGAA
48_ppprot1_063_h09
GGTTTATTTCCAACAATTTTAAGTCTGAGTCTTCAAATGGAGGGGAGTAG
ACATCAGCAGCAAAAACACAGTCTTTCAGATCTTATACCAGTAATTGACC
TTGCAGCACTAAATGGCGATCATATCGACGAATTTGAACGCAGGCGCATT
ATAACTGAGATAGCTCATGCATGCAAAACATGGGGCGCTTTCCAGCTGGT
CAACCATGGTATTCAACCGCATGTGATTGAAAGGGCCAGAGCTAAAGCGT
GCGGGGTGTTTGAATTGCCAAATGAAACACGGTGGAAGGNCAAACGATCA
CCAGGCAGCTTGTCTGGATATGGAAACGGCGCTGTCATCGCACACGCAGT
CAACAATGAAATTGCATCCGAAGCCATAACCTTCGCGTACCAAATTCTG
Auxin metabolism
73_bd05_e04rev
AAAATCCATGAGTCTCCCGAATTAGGCTTTCAAGAATACGGCACTAGTGA
ATTGATAAGAGCAGAGCTTGACCAGATTGGCGTCGATTACACATGGCCGG
TGGCGGAAACCGGCGTTGTAGCCACCATTGGTTCCGGCGAACAACCGTTC
TTTGCTCTCCGTGCCGACATGGATGCTCTTCCACTCCAGGAATTAGTCGA
TTGGGATCACCGGAGTAAGATCGCCGGAAAGATGCACGCCTGCGGTCACG
ATTCTCACGTGACAATGCTACTTGGAGCCGCTAAATTGCTTCAAGCCAAA
AGACATGAACTGAAGGGCACCGTTAAGCTGGTTTTCCAGCCCGGTGAAGA
GGGATTCGCCGGAGCTTACCATATGCTAAAGCATAGTGCACTTGACAACA
TC
47_bd01_h03rev
GCGAATGCTCATAGTCTCAAATCCAGTCTCCTGTGGACCCAATCAGCTGC
AGCGTATCATATTTATTATTACTACTTTAGCAGCTATGAAAGCTTAATAT
CCGGTATCAACAACAACAACCCTACTTTTCCGATCAGTTTACCCGGTCTA
CCGCCACTAACCACGGCGGAGCTACCGTGTATTTTCTTACCTTCGAGACC
CAAGGAACACGATTTTTTTATCCCACTTTCGAAAGACCATATTGATATAC
TTAAAATATCTCCAAGAATACTTGTTAACACTTTTAACGAACTAGAAACT
GAATCCATTACCACATTAGTCCATAAAGTTGAGGTCCTTCCAATTGGTCC
TTTAATGCCATTAGACTCATCGGAAGAT

Appendix B

Cell cycle control
54_ppprot1_52
Pro Ile Arg Glu Lys Tyr Arg Asn His Lys Phe Lys Cys Val Ala Thr
Leu Thr Pro Pro Ser Val Leu Pro Pro Glu Phe Phe Lys Asp Ala Glu
Cys Cys
38_ck7_g07fwd
Thr Asn Arg Asp Val Asn Tyr Asp Ile Thr Pro Val Thr Ala Leu Cys
Val Gly Thr Ala Thr Trp Glu Ser Cys Asn Lys Thr Met Thr Val Lys
Thr Gly Ser Asp Leu Val Cys Arg His Arg Tyr His Ser Lys Lys Tyr
Ala Glu Ile Lys Thr Ser Glu Ser Leu Leu Arg Thr Lys Glu Arg Pro
Lys Ser Thr Thr Pro His
48_ck9_h09fwd
Gly Thr Ser Val Pro Leu Leu Asn Ala Leu Val Leu Tyr Val Gly Met
Gln Ala Val Gln Gln Leu His Ser Lys Thr Ser Gln Gln Leu Ala Ala
Leu Thr Ala Pro Ile Thr His Ser Ala Pro Met Asp Ile Phe Gln Arg
Leu Val Asn Asp Leu Asp Thr Gly Gly Arg Tyr Leu Phe Leu Asn Ala
Val Ala Asn Gln Leu Arg Tyr Pro Asn Asn His Thr Tyr Tyr Phe Ser
Cys Val Leu Leu Phe Leu Phe Ala Glu Ala Ser Leu Glu Ile Ile Gln
Glu Gln Ile Thr Arg Val Leu Leu Glu Arg Leu Ile Val Asn Arg Pro
His Pro Trp Gly Leu Leu Ile Thr Phe Ile Glu Leu Ile Lys Asn Pro
Arg Tyr Ser Phe Trp Thr His Gly Phe Thr Arg Cys Ala Pro Glu Ile
Asp Lys Leu Phe Glu Ser Val Ala Arg Ser Cys Met Asn Ser Thr Leu
Lys Pro Ser Asp Asp Asp Leu Pro Gly Asn Leu Pro Ala Asp Gly Leu
Lys Gly
83_ck6_f06fwd
Asp Ile Ile Asp Ser Ala Leu Leu Arg Pro Gly Arg Leu Asp Gln Leu
Ile Tyr Ile Pro Leu Pro Asp Glu Ala Ser Arg Leu Arg Ile Phe Gln
Ala Ala Leu Arg Lys Ser Pro Leu Ala Lys Glu Val Asp Leu Glu Ala
Leu Ala Arg Tyr Thr Gln Gly Phe Ser Gly Ala Asp Ile Thr Glu Ile
Cys Gln Arg Ala Cys Lys Tyr Ala Ile Arg Glu Asn Ile Glu Lys Asp
Ile Glu Arg Glu Lys Arg Met Ala Glu Asn Pro Glu Ala Met Glu Glu
Asp Glu Val Glu Glu Val Ala Gln Ile Lys Ala Ser His Phe Glu Glu
Ala Met Lys Tyr Ala Arg Arg Ser Val Ser Asp Ala Asp Ile Arg Lys
Tyr Gln Ala Phe Ala Gln Thr Leu Gln Gln Ser Arg Gly Phe Gly Ser
Glu Phe Arg Phe Pro Asp Arg Ala Val Gly Ala Gly Ala Pro Ser Ala
Ala Asp Thr Thr Pro Gly Phe Gly Val Ala Ala Ala Ala Asp Asp Asp
Asp Leu Tyr Ser
06_mm09_a09rev
His Gln Glu His Pro Glu Lys Phe Glu Lys Phe Gly Met Ser Pro Ser
Lys Gly Val Leu Phe Tyr Gly Pro Pro Gly Cys Gly Lys Thr Leu Leu
Ala Lys Ala Ile Ala Asn Glu Cys Gln Ala Asn Phe Ile Ser Val Lys
Gly Pro Glu Leu Leu Thr Met Trp Phe Gly Glu Ser Glu Ala Asn Val
Arg Asp Val Phe Asp Lys Ala Arg Gln Ser Ala Pro Cys Val Leu Phe
Phe Asp Glu Leu Asp Ser Ile Ala Asn Gln Arg Gly Ser Ser Gln Gly
Asp Ala Gly Gly Ala Ala Asp Arg Val Leu Asn Gln Leu Leu Thr Glu
Met Asp Gly Met Asn Ala Lys Lys Thr Val Phe Ile Ile Gly Ala Thr
Asn Arg Pro Asp Ile Ile Asp Ser Ala Leu Leu Arg Pro Gly Arg Leu
Gly Ser Ala His Leu His Ser Pro Xaa Xaa
71_ck3l_d06fwd
Ala Arg Ala Glu Leu Gln Gln Leu Leu His Ile Phe Arg Leu Leu Gly
Thr Pro Asn Glu Thr Ile Trp Pro Gly Val Ser Gln His Arg Asp Trp
His Glu Phe Pro Gln Trp Arg Pro Gln Asp Leu Ser Leu Ala Val Pro
Gly Leu Ser Ala Val Gly Leu Asp Leu Leu Ala Lys Met Leu Val Phe
Glu Pro Ser Lys Arg Ile Ser Ala Eqs Ala Ala Leu Ser His Thr Tyr
Phe Ala Asp Val Asp Lys Thr Ala Thr
54_ppprot3_003_a12
Ser Arg Tyr Leu Lys Phe Gln Met Asp Thr Tyr Glu Lys Val Glu Lys
Ile Gly Glu Gly Thr Xaa Gly Val Val Tyr Lys Ala Arg Asp Arg Leu
Thr Asn Glu Thr Ile Ala Leu Lys Lys Ile Arg Leu Glu Gln Glu Asp
Glu Gly Val Pro Ser Xaa Ala Ile Arg Xaa Ile Ser Leu Leu Lys Glu
Met His His Gly Asn Ile Val Arg Leu Gln Asp Val Val
Cytokinin metabolism
06ppprot1_062_a09
Phe Leu Met Glu Cys Arg Gln Val Glu Gly Ala Ala Thr Glu Glu Arg
Phe Phe Leu Phe Leu Glu Glu Phe Gln Arg His Ser Arg Asn Tyr Val
Lys Arg Gln Leu Thr Trp Phe Arg Asn Lys Gly Gln Ser Glu Gln Met
Phe Asn Trp Ile Asp Ala Thr Gln Pro Leu Glu Val Met Val Asp Ala
Leu Ala Lys Glu Tyr Glu Arg Pro Asn Glu Val Val Ser Asp Val Leu
Lys Ala Ala Ser Val Val Thr Lys Glu Ser Ser Tyr Lys Glu Glu Asn
Leu Leu Lys Arg Tyr Arg Thr Gln Asn Arg Ile Phe Thr Ser Asn Ser
Glu Ala Leu Lys Arg Thr Leu Gln Trp Ile Arg Asp Thr Gln Cys Leu
Trp Arg Asn Ser Ser Thr Val Asp Asp Leu Gln Lys Arg Met Glu Ser
Ser Leu Thr Thr Ser Met
Steroid metabolism
36_ck24_f09fwd
Ile Glu Arg Glu Arg Glu Arg Glu Arg Glu Arg Gln Glu Leu Thr Pro
Ala Trp Gly Ala Val Ser Thr Ser Ser Ile Ser Met Ala Thr Ala Leu
Glu Lys Gly Trp Leu Tyr Leu Ile Asn Asn Phe Thr Asp Phe Gln Leu
Ala Ser Ile Gly Ser Phe Ile Ile His Glu Ser Val Phe Phe Leu Ser
Gly Leu Pro Phe Ile Val Met Glu Arg Leu Gly Tyr Gln Arg Gln Tyr
Lys Ile Gln Gly Lys Val Asn Ser Val Ala Ala Gln Glu Lys Cys Val
Met Lys Leu Leu Ile Tyr His Ile Cys Val Asn Leu Pro Leu Met Ile
Val Ser Tyr Pro Val Phe Lys Tyr Met Gly Phe Thr Ser Gln Leu Pro
Leu Pro Ser Trp Asn Val Val Cys Phe Gln Ile Tyr Leu Ile Leu Ser
02_ppprot1_087_a07
Ala Arg Gly Ser Phe Pro Ala Val Arg Leu Ile Gly Val Arg Phe Gly
Leu Pro Leu Pro Ala Val Ser Glu Val Leu Met Gln Leu Thr Val Tyr
Thr Ile Val Glu Asp Phe Gly Asn Tyr Trp Leu His Arg Trp Leu His
Asn Gly Trp Trp Tyr Asp Ala Ile His Ser Val His His Glu Phe Ser
Ala Pro Met Ser Phe Ala Ala Pro Tyr Ala His Trp Ala Glu Val Val
Ile Leu Gly Val Pro Thr Phe Ala Gly Pro Ala Met Ala Pro Gly His
Ile Ile Thr Phe Trp Leu Trp Ile Ala Met Arg Gln Leu Glu Ala Leu
Glu Thr His Ser Gly Tyr Asp Phe Pro Trp Asn Pro Thr Arg Leu Ile
Pro Phe Tyr Gly Gly Ala Glu Tyr His Asp Tyr His His Phe Val Gly
Leu Tyr Gly Thr Asp Lys Gly Tyr Arg Tyr Met Lys Glu Val His Arg
Lys
26_ppprot1_054_e07
Ala Arg Gly Gly Val Lys His Leu Ala Pro Ala Cys Ser His Ser His
Ala Pro Pro Arg Phe Ser Leu Leu Asn Pro Cys Ala Ser Thr Val Cys
Ser Pro Gly Leu Glu Tyr Gly Phe Ser Gly Trp Val Pro Ser His Cys
Phe Pro Arg Pro Thr Leu Phe His Arg Pro Arg Val Val Trp His Phe
Thr Ala Ala Ala Leu Cys Val Val Leu Phe Pro Ser Ala Ser Thr Glu
Lys Leu Ala Asp Met Ala Lys Glu Gly Ala Arg His Leu Val Ser Asn
Ile Gly Gly Val Leu Pro Lys His Glu Val Leu Lys Ser Thr Glu Glu
Tyr Glu Lys Tyr His Ala Met His Gly Gly Asp Lys Glu Ala Arg Lys
Ser Asn Tyr Thr Asp Met Val Asn Lys Tyr Tyr Asp Leu Val Asn Ser
Phe Tyr Glu Tyr Gly Trp Gly Glu Ser Tyr His Phe Ala Asn Arg Trp
Arg Gly Glu Thr Leu Arg Glu Ser Ile Lys Arg His Glu His Phe Leu
Ala Leu His Leu His Leu Lys Pro Gly Met Lys Val Leu Asp Val Gly
Cys Gly Ile Gly Gly Pro Ala Arg Gly Ile
Inositolephosphate-dependent Pathway
88_ppprot135_g11
Val Ala Ile Arg Glu Leu Ser Gln Leu Arg Thr Gln Gly Ser Cys Pro
Pro Lys Asp Gly Phe Thr Asn Met Gly Leu Gln Gln Cys Glu Asp Leu
Glu Thr Cys Leu Ala Val Ala Val Asp Val Ala Lys Lys Ala Gly Gln
Ile Ile Lys Asp Gly Phe His Ile Ala Lys Ala Val Glu His Lys Gly
Met Val Asp Leu Val Thr Glu Thr Asp Lys Ala Cys Glu Asp Leu Ile
Phe Thr Gln Leu Lys Thr Ser Phe Pro Ser His Glu Leu Ile Gly Glu
Glu Glu Ser Ser Glu Ser Gly Ile Pro Leu Leu Thr Asp Ala Pro Thr
Trp Val Val Asp Pro Leu Asp Gly Thr Thr Asn Phe Val His Lys Phe
Pro Phe Val Cys Val Ser Ile Gly Leu Val Ile Asn Lys Val
87_mm12_g05rev
His Glu Glu Thr Asn Leu Glu Leu Phe Lys Tyr Tyr Thr Asp Thr Ser
Arg Gly Val Arg Arg Leu Gly Ala Ala Ala Val Asp Cys Cys His Val
Ala Leu Gly Ile Ala Asp Ser Tyr Trp Glu Phe Arg Leu Lys Pro Trp
Asp Met Ala Ala Gly Ala Leu Met Val Glu Glu Ala Gly Gly Lys Val
Thr Arg Met Asp Gly Gly Pro Phe Ser Val Phe Asp Arg Ser Val Ile
Val Ser Asn Gly Ala Ile His Asp Lys Leu Leu Glu Lys Ile Gln Pro
Ala Thr Glu Lys Leu Ile Ala Asp Gly Leu Asn Phe Ser Gln Trp Leu
Lys Pro Thr Gly Tyr Asn Ser Asp Val
Phytochrome
53_mm18_a06rev
Ala Pro Val Asn Asp Phe Arg Asn Lys Lys Tyr Ala Pro Gly Ala Val
Thr Pro Phe Ser Ile Thr Gln Ala Val Asp Leu Met Leu Gln Leu Ala
Glu Gly Val Arg Tyr Leu His Ser Lys His Leu Ala His Arg Asp Ile
Lys Ser Gly Asn Val Leu Leu Gln Phe Ala Asp Pro Lys His Gly Thr
Thr Glu Pro Trp Ser Asn Gly Asn Thr Cys Pro Phe Ile Ala Lys Val
Ala Asp Phe Gly Leu Thr Lys Ile Lys Asn Thr Ser Thr His Arg Gly
His Gln Thr Leu Met Thr Gly Thr Arg Pro Trp Met Ala Pro Glu Ala
Tyr Lys Tyr Glu Trp Thr Asp Glu Pro Thr Pro Ser Ser Arg Tyr His
Pro Met Lys Leu Asp Val Tyr Gly Phe Gly Ile Met Cys Cys Glu Ile
Leu Ser Gly Glu Glu Pro Tyr Gln Lys Leu Pro Ser Tyr Ala Ala Val
Lys Ala Gly Glu Arg Pro Glu Val Ala
Gibberellin metabolism
93_ck24_h05fwd
Gly Thr Ser Asp Tyr Leu Asn Gln Leu Leu Ile Lys Phe Asp His Ala
Cys Pro Asn Val Tyr Pro Val Asp Leu Phe Glu Arg Leu Trp Met Val
Asp Arg Leu Gln Arg Leu Gly Ile Ser Arg Tyr Phe Glu Arg Glu Ile
Arg Asp Cys Leu Gln Tyr Val Tyr Arg Tyr Trp Lys Asp Cys Gly Ile
Gly Trp Ala Ser Asn Ser Ser Val Gln Asp Val Asp Asp Thr Ala Met
Ala Phe Arg Leu Leu Arg Thr His Gly Phe Asp Val Lys Glu Asp Cys
Phe Arg Gln Phe Phe Lys Asp Gly Glu Phe Phe Cys Phe Ala Gly Gln
Ser Ser Gln Ala Val Thr Gly Met Phe Asn Leu Ser Arg Ala Ser Gln
Thr Leu Phe Pro Gly Glu Ser Leu Leu Lys Lys Ala Xaa Thr Phe Ser
Arg Asn Phe Leu Arg Thr Lys His Glu Asn Asn Glu Cys Phe Asp Lys
Trp
51_ppprot1_0052_a05
Lys Arg Glu Glu Asn Glu Lys Ser Arg Ile Pro Met Ala Met Val Tyr
Lys Tyr Pro Thr Thr Leu Leu His Ser Leu Glu Gly Leu His Arg Glu
Val Asp Trp Asn Lys Leu Leu Gln Leu Gln Ser Glu Asn Gly Ser Phe
Leu Tyr Ser Pro Ala Ser Thr Ala Cys Ala Leu Val His Lys Arg Cys
Glu Val Leu Arg Leu Leu Glu Pro Ala Pro His Gln Val Arg Pro Arg
Leu Ser Lys Arg Val Pro Arg
38_ppprot1_046_g07
Ala Arg Gly Leu Glu His Arg Thr Tyr Leu Asp Gln Tyr Gly Ile Asp
Asp Ile Trp Ile Gly Lys Ser Leu Tyr Lys Met Pro Ala Val Thr Asn
Glu Val Phe Leu Lys Leu Ala Lys Ala Asp Phe Asn Met Cys Gln Ala
Leu His Lys Lys Glu Leu Glu Gln Val Ile Lys Trp Asn Ala Ser Cys
Gln Phe Arg Asp Leu Glu Phe Ala Arg Gln Lys Ser Val Gia Cys Tyr
Phe Ala Gly Ala Ala Thr Met Phe Glu Pro Glu Met Val Gln Ala Arg
Leu Val Trp Ala Arg Cys Cys Val Leu Thr Thr Val Leu Asp Asp Tyr
Phe Asp His Gly Thr Pro Val Glu Glu Leu Arg Val Phe Val Gln Ala
Val Arg Thr Trp Asn Pro Glu Leu Ile Asn Gly Leu Pro Glu Gln Ala
Lys Ile Leu Phe Met Gly Leu Tyr Lys Thr Val Asn Thr Ile Ala Glu
Glu Ala Phe Met Ala Gln Lys Arg Asp Val His His His Leu Lys His
Tyr Trp Asp Lys Leu Ile Thr Ser Ala Leu Lys Glu Ala Arg Met Gly
Arg Val Arg Leu Arg Ser Pro Pro Ser Thr Ser Ile Trp Lys Ser Leu
66_ppprot1_63
Phe Thr Ala Val Pro Lys Ser Cys Lys Arg Ile His Leu Asn Met Ala
Lys Ile Met His Ala Phe Tyr Lys Asp Thr Asp Gly Phe Ser Ser Leu
Thr Ala Met Thr Gly Phe Val Lys Lys Val Leu Phe Glu Pro Val Pro
Glu
48_ppprot1_063_h09
Gly Leu Phe Pro Thr Ile Leu Ser Leu Ser Leu Gln Met Glu Gly Ser
Arg His Glu Gln Gln Lys Gln Ser Leu Ser Asp Leu Ile Pro Val Ile
Asp Leu Ala Ala Leu Asn Gly Asp His Ile Asp Glu Phe Glu Arg Arg
Arg Ile Ile Thr Glu Ile Ala His Ala Cys Lys Thr Trp Gly Ala Phe
Gln Leu Val Asn His Gly Ile Gln Pro His Val Ile Glu Arg Ala Arg
Ala Lys Ala Cys Gly Val Phe Glu Leu Pro Asn Glu Thr Arg Trp Lys
Xaa Lys Arg Ser Pro Gly Ser Leu Ser Gly Tyr Gly Asn Gly Ala Val
Ile Ala Asp Ala Val Asn Asn Glu Ile Ala Ser Glu Ala Ile Thr Phe
Gly Tyr Gln Ile Leu
Auxin metabolism
73_bd05_e04rev
Lys Ile His Glu Ser Pro Glu Leu Gly Phe Gln Glu Tyr Gly Thr Ser
Glu Leu Ile Arg Ala Glu Leu Asp Gln Ile Gly Val Asp Tyr Thr Trp
Pro Val Ala Glu Thr Gly Val Val Ala Thr Ile Gly Ser Gly Glu Gln
Pro Phe Phe Ala Leu Arg Ala Asp Met Asp Ala Leu Pro Leu Gln Glu
Leu Val Asp Trp Asp His Arg Ser Lys Ile Ala Gly Lys Met His Ala
Cys Gly His Asp Ser His Val Thr Met Leu Leu Gly Ala Ala Lys Leu
Leu Gln Ala Lys Arg His Glu Leu Lys Gly Thr Val Lys Leu Val Phe
Gln Pro Gly Glu Glu Gly Phe Ala Gly Ala Tyr His Met Leu Lys His
Ser Ala Leu Asp Asn Ile
47_bd01_h03rev
Ala Asn Ala His Ser Leu Lys Ser Ser Leu Leu Trp Thr Gln Ser Ala
Ala Ala Tyr His Ile Tyr Tyr Tyr Tyr Phe Ser Ser Tyr Glu Ser Leu
Ile Ser Gly Ile Asn Asn Asn Asn Pro Thr Phe Pro Ile Ser Leu Pro
Gly Leu Pro Pro Leu Thr Thr Ala Glu Leu Pro Cys Ile Phe Leu Pro
Ser Arg Pro Lys Glu His Asp Phe Phe Ile Pro Leu Ser Lys Asp His
Ile Asp Ile Leu Lys Ile Ser Pro Arg Ile Leu Val Asn Thr Phe Asn
Glu Leu Glu Thr Glu Ser Ile Thr Thr Leu Val His Lys Val Glu Val
Leu Pro Ile Gly Pro Leu Met Pro Leu Asp Ser Ser Glu Asp

1. An isolated nucleic acid molecule from a moss encoding a Growth Development Related Protein (GDRP), or a portion thereof.

2. An isolated nucleic acid molecule wherein the moss is selected from Physcomitrella patens or Ceratodon purpureus.

3. The isolated nucleic acid molecule of claim 1 or 2, wherein said nucleic acid molecule encodes an GDRP involved in the regulation of cell division, growth and biomass formation.

4. The isolated nucleic acid molecule of any one of claims 1 to 3, wherein said nucleic acid molecule encodes an GDRP involved in the metabolism of phytohormones, signaling pathways and/or photoreceptor systems.

5. The isolated nucleic acid molecule of any one of claims 1 to 4, wherein said nucleic acid molecule encodes an GDRP involved in the metabolism of auxins, brassino-steroids, cytokinins and/or gibberellins and/or the inositolphosphat-dependent signaling pathway and/or the phytochrome photoreceptor system.

6. The isolated nucleic acid molecule of any one of claims 1 to 5, wherein said nucleic acid molecule encodes an GDRP involved in the regulation of the plant cell cycle.

7. An isolated nucleic acid molecule from mosses selected from the group consisting of those sequences set forth in Appendix A, or a portion thereof.

8. An isolated nucleic acid molecule which encodes a polypeptide sequence selected from the group consisting of those sequences set forth in Appendix B.

9. An isolated nucleic acid molecule which encodes a naturally occurring allelic variant of a polypeptide selected from the group of amino acid sequences consisting of those sequences set forth in Appendix B.

10. An isolated nucleic acid molecule comprising a nucleotide sequence which is at least 50% homologous to a nucleotide sequence selected from the group consisting of those sequences set forth in Appendix A, or a portion thereof.

11. An isolated nucleic acid molecule comprising a fragment of at least 15 nucleotides of a nucleic acid comprising a nucleotide sequence selected from the group consisting of those sequences set forth in Appendix A.

12. An isolated nucleic acid molecule which hybridizes to the nucleic acid molecule of any one of claims 1-11 under stringent conditions.

13. An isolated nucleic acid molecule comprising the nucleic acid molecule of any one of claims 1-12 or a portion thereof and a nucleotide sequence encoding a heterologous polypeptide.

14. A vector comprising the nucleic acid molecule of any one of claims 1-13.

15. The vector of claim 14, which is an expression vector.

16. A host cell transformed with the expression vector of claim 15.

17. The host cell of claim 16, wherein said cell is a microorganism.

18. The host cell of claim 16, wherein said cell belongs to the genus mosses or algae.

19. The host cell of claim 16, wherein said cell is a plant cell.

20. The host cell of any one of claims 16 to 19, wherein the expression of said nucleic acid molecule results in the modulation of the production of biomass from the said cell.

21. The host cell of any one of claims 16 to 20, wherein the expression of said nucleic acid molecule results in an increased production of biomass from said cell.

22. The host cell of any one of claims 16 to 21, wherein the expression of said nucleic acid molecule results in an improved relation of apical growth and root formation from said cell.

23. Descendants, seeds or reproducable cell material derived from a host cell of any one of claims 16 to 22.

24. A method of producing a polypeptide comprising culturing the host cell of any one of claims 16 to 22 under appropriate culture conditions to, thereby, produce the polypeptide.

25. An isolated GDR polypeptide from mosses or algae or a portion thereof.

26. An isolated GDR polypeptide from microorganisms or fungi or a portion thereof.

27. An isolated GDR polypeptide from plants or a portion thereof.

28. The polypeptide of any one of claims 25 to 27, wherein said polypeptide is involved in the metabolism of phytohormones, signaling pathways, photoreceptor systems and/or the regulation of the plant cell cycle.

29. The polypeptide of any one of claims 25 to 28, wherein said polypeptide is involved in the metabolism of auxins, brassino-steroids, cytokinins and/or gibberellins and/or the inositolphosphat-dependent signaling pathway and/or the phytochrome photoreceptor system.

30. An isolated polypeptide comprising an amino acid sequence selected from the group consisting of those sequences set forth in Appendix B.

31. An isolated polypeptide comprising a naturally occurring allelic variant of a polypeptide comprising an amino acid sequence selected from the group consisting of those sequences set forth in Appendix B, or a portion thereof.

32. The isolated polypeptide of any of claims 25 to 31, further comprising heterologous amino acid sequences.

33. An isolated polypeptide which is encoded by a nucleic acid molecule comprising a nucleotide sequence which is at least 50% homologous to a nucleic acid selected from the group consisting of those sequences set forth in Appendix A.

34. An isolated polypeptide comprising an amino acid sequence which is at least 50% homologous to an amino acid sequence selected from the group consisting of those sequences set forth in Appendix B.

35. An antibody specifically binding to a GDR polypeptide of any one of claims 25 to 34 or a portion thereof.

36. Test kit comprising a nucleic acid molecule of any one of claims 1 to 13, a portion and/or a complement thereof used as probe or primer for identifying and/or cloning further nucleic acid molecules involved in the growth development.

37. Test kit comprising an GDR polypeptide-antibody of claim 35 for identifying and/or purifying further GDR polypeptide molecules or fragments thereof in other cell types or organisms.

38. A method for producing high yield plants, comprising culturing a cell containing a vector of claim 14 or 15 such that a high yield plant phaenotype is produced.

39. The method of claim 38, wherein said method further comprises the step of recovering the expression products selected from the group of phytohormones or photoreceptors from said cell.

40. The method of claim 38 or 39, wherein said method further comprises the step of transforming said cell with the vector of claim 14 or 15 to result in a cell containing said vector.

41. The method of any one of claims 38 to 40, wherein said cell is a microorganism.

42. The method of any one of claims 38 to 40, wherein said cell belongs to the genus mosses or algae.

43. The method of any one of claims 38 to 40, wherein said cell is a plant cell.

44. The method of any one of claims 38 to 43, wherein expression of the nucleic acid molecule from said vector results in modulation of the production of biomass.

45. The method of claim 44, wherein said modulation of biomass production is an improved relation of apical growth and root formation.

46. A method for improving the production of total plant biomass, comprising culturing a cell whose genomic DNA has been altered by the inclusion of a nucleic acid molecule of any one of claims 1-13.

47. A high yield plant produced by a method of any one of claims 25 to 34.

48. Use of a high yield plant of claim 47 or a polypeptide of any one of claims 22 to 34 for the production of another high yield plant.
